SEQUENCE LISTING (25 sequences)

SEQ ID NO: 1 - protein, 103 aa, synthetic construct
QVQLVESGGGLVQPGGSLRLSCAASSLGWFRQAPGQGLEAVAAIASMGGLKYYADSVKGRFTISRDNSKNTLYLQMNSLRAEDTAVYYCAARYWGQGTLVTVS

SEQ ID NO: 2 - protein, 14 aa, synthetic construct
MFYPDSRCRGPSET

SEQ ID NO: 3 - protein, 16 aa, synthetic construct
CMFYPDSRCRGPSETC

SEQ ID NO: 4 - protein, 7 aa, synthetic construct
MFYPDSR

SEQ ID NO: 5 - protein, 7 aa, synthetic construct
CRGPSET

SEQ ID NO: 6 - protein, 9 aa, synthetic construct
CMFYPDSRC

SEQ ID NO: 7 - protein, 8 aa, synthetic construct
CRGPSETC

SEQ ID NO: 8 - protein, 14 aa, synthetic construct
MFYPDSRSRGPSET

SEQ ID NO: 9 - protein, 19 aa, synthetic construct
MDWTWRVFCLLAVAPGAHS

SEQ ID NO: 10 - protein, 26 aa, synthetic construct
MLGKNDPMCLVLVLLGLTALLGICQG

SEQ ID NO: 11 - protein, 131 aa, synthetic construct
QVQLVESGGGLVQPGGSLRLSCAASMFYPDSRCRGPSETSLGWFRQAPGQGLEAVAAIASMGGLKYYADSVKGRFTISRDNSKNTLYLQMNSLRAEDTAVYYCAAVRGYFMRLPSSHNFRYWGQGTLVTVS

SEQ ID NO: 12 - protein, 13 aa, synthetic construct
AEDTAVYYCAAVR

SEQ ID NO: 13 - protein, 131 aa, synthetic construct
QVQLVESGGGLVQPGGSLRLSCAASMFYPDSRCRGPSETSLGWFRQAPGQGLEAVAAIASMGGLKYYADSVKGRFTISRDNSKNTLYLOMNSLRAEDTAVYYCAAVRGYFMRLPSSHNFRYWGOGTLVTVS

SEQ ID NO: 14 - DNA, 468 bp, synthetic construct
  source 1..468: mol_type = other DNA
gccaccatgcatctaatggattggacttggcgagtgttttgcctgctggccgtggctcctggcgctcactctcaggtgcagctggtcgagtccggaggaggcctggtgcagcctggcggctccctgagactgtcttgtgctgcttccatgttctatcctgattctagatgcagaggcccctccgagacatctctgggctggttcagacaggccccaggccagggcctggaagctgtggccgccatcgcctctatgggcggactgaagtactacgccgactccgtgaaaggcagattcaccatctcccgcgacaactccaagaacaccctgtacctgcagatgaatagcctgcgggccgaggacaccgccgtgtactactgcgccgctgtgcggggctacttcatgcggctgcctagctctcacaacttccggtactggggccaaggcaccctcgtgaccgtgtcctga

SEQ ID NO: 15 - protein, 153 aa, synthetic construct
MHLMDWTWRVFCLLAVAPGAHSQVQLVESGGGLVQPGGSLRLSCAASMFYPDSRCRGPSETSLGWFRQAPGQGLEAVAAIASMGGLKYYADSVKGRFTISRDNSKNTLYLQMNSLRAEDTAVYYCAAVRGYFMRLPSSHNFRYWGQGTLVTVS

SEQ ID NO: 16 - DNA, 444 bp, synthetic construct
  source 1..444: mol_type = other DNA
ttcgaaatgcaccatcatcaccaccatgatgatgacgataagcaggtgcagctggtggaaagcggcggcggcctggtgcagccgggcggcagcctgcgcctgagctgcgcggcgagcatgttttatccggatagccgctgccgcggcccgagcgaaaccagcctgggctggtttcgccaggcgccgggccagggcctggaagcggtggcggcgattgcgagcatgggcggcctgaaatattatgcggatagcgtgaaaggccgctttaccattagccgcgataacagcaaaaacaccctgtatctgcagatgaacagcctgcgcgcggaagataccgcggtgtattattgcgcggcggtgcgcggctattttatgcgcctgccgagcagccataactttcgctattggggccagggcaccctggtgaccgtgagctaacatatg

SEQ ID NO: 17 - protein, 143 aa, synthetic construct
MHHHHHHDDDDKQVQLVESGGGLVQPGGSLRLSCAASMFYPDSRCRGPSETSLGWFRQAPGQGLEAVAAIASMGGLKYYADSVKGRFTISRDNSKNTLYLQMNSLRAEDTAVYYCAAVRGYFMRLPSSHNFRYWGQGTLVTVS

SEQ ID NO: 18 - DNA, 521 bp, synthetic construct
  source 1..521: mol_type = other DNA
ttcgaaatgctgggcaaaaacgatccgatgtgcctggtgctggtgctgctgggcctgaccgcgctgctgggcatttgccagggccaccatcatcaccaccatgatgatgacgataagcaggtgcagctggtggaaagcggcggcggcctggtgcagccgggcggcagcctgcgcctgagctgcgcggcgagcatgttttatccggatagccgctgccgcggcccgagcgaaaccagcctgggctggtttcgccaggcgccgggccagggcctggaagcggtggcggcgattgcgagcatgggcggcctgaaatattatgcggatagcgtgaaaggccgctttaccattagccgcgataacagcaaaaacaccctgtatctgcagatgaacagcctgcgcgcggaagataccgcggtgtattattgcgcggcggtgcgcggctattttatgcgcctgccgagcagccataactttcgctattggggccagggcaccctggtgaccgtgagctgagcggccgc

SEQ ID NO: 19 - protein, 168 aa, synthetic construct
MLGKNDPMCLVLVLLGLTALLGICQGHHHHHHDDDDKQVQLVESGGGLVQPGGSLRLSCAASMFYPDSRCRGPSETSLGWFRQAPGQGLEAVAAIASMGGLKYYADSVKGRFTISRDNSKNTLYLQMNSLRAEDTAVYYCAAVRGYFMRLPSSHNFRYWGQGTLVTVS

SEQ ID NO: 20 - protein, 122 aa, synthetic construct
QVQLVESGGGVVQPGKSLRLSCAASGFTFSGYGMHWVRQAPGKGLEWVALISYDESNKYYADSVKGRFTISRDNSKNTLYLQMNSLRAEDTAVYYCAKVKFYDPTAPNDYWGQGTLVTVSSG

SEQ ID NO: 21 - protein, 131 aa, synthetic construct
QVQLVESGGGLVQPGGSLRLSCAASMFYPDSRCRGPSETSLGWFRQAPGQGLEAVAAIASMGGLTYYADSVKGRFTISRDNSKNTLYLQMNSLRAEDTAVYYCAAVRGYFMRLPSSHNFRYWGQGTLVTVS

SEQ ID NO: 22 - protein, 127 aa, synthetic construct
QVQLVESGGGLVQPGGSLRLSCAASGGSEYSYSTFSLGWFRQAPGQGLEAVAAIASMGGLTYYADSVKGRFTISRDNSKNTLYLQMNSLRAEDTAVYYCAAMFYPDSRCRGPSETRYWGQGTLVTVS

SEQ ID NO: 23 - protein, 131 aa, synthetic construct
QVQLVESGGGLVQPGGSLRLSCAASMFYPDSRCRGPSETSLGWFRQAPGQGLEAVAAIASMGGLTYYADSVKGRFTISRDNSKNTLYLQMNSLRAEDTAVYYCAAMFYPDSRCRGPSETRYWGQGTLVTVS

SEQ ID NO: 24 - protein, 22 aa, synthetic construct
MHLMDWTWRVFCLLAVAPGAHS

SEQ ID NO: 25 - protein, 18 aa, synthetic construct
MKVWTFISLLFLFSSAYS

(19) United States
(12) Patent Application Publication
(10) Pub. No.: US 2026/0041783 A1
(43) Pub. Date: Feb. 12, 2026

(54) Nanobody platform, Peptide-modified Nanobody, Production Process, and Use

(71) Applicant: PHP BIOTECH INTERNATIONAL INC., Keller, TX (US)

(72) Inventors: Moacyr Ramos BIGHETTI, São Paulo (BR); Patricia Heloise Alves BEZERRA, São Paulo (BR)

(73) Assignee: PHP BIOTECH INTERNATIONAL INC., Keller, TX (US)

(21) Appl. No.: 19/223,678

(22) Filed: May 30, 2025

Related U.S. Application Data
(60) Provisional application No. 63/653,708, filed on May 30, 2024.

(51) Int. Cl. A61K 47/68; A61K 38/00; A61P 35/00; C07K 14/46; C07K 16/12; C12N 15/70; C12N 15/81; C12N 15/85

(52) U.S. Cl. CPC A61K 47/6811; A61K 47/6835; A61P 35/00; C07K 14/46; C07K 16/1271; C12N 15/70; C12N 15/815; C12N 15/85; A61K 38/00; C07K 2317/14; C07K 2317/24; C07K 2317/569; C07K 2319/00

(57) ABSTRACT

The present disclosure provides a nanobody platform having a high affinity purification on protein A, wherein the nanobodies may incorporate diverse bioactive peptides, lipid or glycoside in its CDR1 and/or CDR3 regions. The present disclosure also provides a peptide-modified nanobody comprising non-toxic bioactive peptides and that exhibit potent anti-tumor activity. Moreover, the peptide-modified nanobody is able to be combined with other technologies, such as but not limited to bispecific antibodies and antibody-drug conjugates. Further, the present disclosure also provides production processes of the nanobodies and their use as a treatment and diagnostic agents.

### CROSS-REFERENCE TO RELATED APPLICATION

This application is related, and claims priority, to U.S. provisional patent application Ser. No. 63/653,708, filed May 30, 2024, the contents of which are incorporated by reference herein as of fully written herein.

### STATEMENT CONCERNING SEQUENCE LISTING

This application includes sequence listings in the application text and Figures. The sequence listings are set forth in the accompanying .xml file named 0016788USU_1539_Sequence Listing.xml. This file was created on Oct. 9, 2025, and contains 25,216 bytes. These sequence listings were all disclosed in the application as filed; thus the file named 0016788USU_1539_Sequence Listing.xml contains no new matter. For ease of reference the following list of the location of each sequence is set forth as follows:

Sequence ID 1
QVQLVESGGGLVQPGGSLRLSCAASSLGWFRQAPGQGLEAVAAIASMGGLKYYADSV
KGRFTISRDNSKNTLYLQMNSLRAEDTAVYYCAARYWGQGTLVTVS (Specification
page 45)
Sequence ID 2
MFYPDSRCRGPSET (Specification page 45)
Sequence ID 3
CMFYPDSRCRGPSETC (Specification page 45)
Sequence ID 4
MFYPDSR (Specification page 45)
Sequence ID 5
CRGPSET (Specification page 46)
Sequence ID 6
CMFYPDSRC (Specification page 46)
Sequence ID 7
CRGPSETC (Specification page 46)
Sequence ID 8
MFYPDSRSRGPSET (Specification page 46)
Sequence ID 9
MDWTWRVFCLLAVAPGAHS (Specification page 46)
Sequence ID 10
MLGKNDPMCLVLVLLGLTALLGICQG (Specification page 37)
Sequence ID 11
QVQLVESGGGLVQPGGSLRLSCAASMFYPDSRCRGPSETSLGWFRQAPGQGLEAVA
AIASMGGLKYYADSVKGRFTISRDNSKNTLYLQMNSLRAEDTAVYYCAAVRGYFMRLP
SSHNFRYWGQGTLVTVS (FIG. 2)
Sequence ID 12
AEDTAVYYCAAVR (FIG. 3)
Sequence ID 13
QVQLVESGGGLVQPGGSLRLSCAASMFYPDSRCRGPSETSLGWFRQAPGQGLEAVA
AIASMGGLKYYADSVKGRFTISRDNSKNTLYLQMNSLRAEDTAVYYCAAVRGYFMRLP
SSHNFRYWGQGTLVTVS (FIG. 3)
Sequence 14
IDGCCACCATGCATCTAATGGATTGGACTTGGCGAGTGTTTTGCCTGCTGGCCGTG
GCTCCTGGCGCTCACTCTCAGGTGCAGCTGGTCGAGTCCGGAGGAGGCCTGGTG
CAGCCTGGCGGCTCCCTGAGACTGTCTTGTGCTGCTTCCATGTTCTATCCTGATTC
TAGATGCAGAGGCCCCTCCGAGACATCTCTGGGCTGGTTCAGACAGGCCCCAGGC
CAGGGCCTGGAAGCTGTGGCCGCCATCGCCTCTATGGGCGGACTGAAGTACTACG
CCGACTCCGTGAAAGGCAGATTCACCATCTCCCGCGACAACTCCAAGAACACCCT
GTACCTGCAGATGAATAGCCTGCGGGCCGAGGACACCGCCGTGTACTACTGCGCC
GCTGTGCGGGGCTACTTCATGCGGCTGCCTAGCTCTCACAACTTCCGGTACTGGG
GCCAAGGCACCCTCGTGACCGTGTCCTGA (FIG. 6)
Sequence ID 15
MHLMDWTWRVFCLLAVAPGAHSQVQLVESGGGLVQPGGSLRLSCAASMFYPDSRCR
GPSETSLGWFRQAPGQGLEAVAAIASMGGLKYYADSVKGRFTISRDNSKNTLYLQMNS
LRAEDTAVYYCAAVRGYFMRLPSSHNFRYWGQGTLVTVS (FIG. 6)
Sequence ID 16
TTCGAAATGCACCATCATCACCACCATGATGATGACGATAAGCAGGTGCAGCTGGT
GGAAAGCGGCGGCGGCCTGGTGCAGCCGGGCGGCAGCCTGCGCCTGAGCTGCG
CGGCGAGCATGTTTTATCCGGATAGCCGCTGCCGCGGCCCGAGCGAAACCAGCCT
GGGCTGGTTTCGCCAGGCGCCGGGCCAGGGCCTGGAAGCGGTGGCGGCGATTG
CGAGCATGGGCGGCCTGAAATATTATGCGGATAGCGTGAAAGGCCGCTTTACCATT
AGCCGCGATAACAGCAAAAACACCCTGTATCTGCAGATGAACAGCCTGCGCGCGG
AAGATACCGCGGTGTATTATTGCGCGGCGGTGCGCGGCTATTTTATGCGCCTGCCG
AGCAGCCATAACTTTCGCTATTGGGGCCAGGGCACCCTGGTGACCGTGAGCTAAC
ATATG (FIG. 16)
Sequence ID 17
MHHHHHHDDDDKQVQLVESGGGLVQPGGSLRLSCAASMFYPDSRCRGPSETSLGW
FRQAPGQGLEAVAAIASMGGLKYYADSVKGRFTISRDNSKNTLYLQMNSLRAEDTAVY
YCAAVRGYFMRLPSSHNFRYWGQGTLVTVS (FIG. 16)
Sequence ID 18
TTCGAAATGCTGGGCAAAAACGATCCGATGTGCCTGGTGCTGGTGCTGCTGGGCC
TGACCGCGCTGCTGGGCATTTGCCAGGGCCACCATCATCACCACCATGATGATGAC
GATAAGCAGGTGCAGCTGGTGGAAAGCGGCGGCGGCCTGGTGCAGCCGGGCGG
CAGCCTGCGCCTGAGCTGCGCGGCGAGCATGTTTTATCCGGATAGCCGCTGCCGC
GGCCCGAGCGAAACCAGCCTGGGCTGGTTTCGCCAGGCGCCGGGCCAGGGCCT
GGAAGCGGTGGCGGCGATTGCGAGCATGGGCGGCCTGAAATATTATGCGGATAGC
GTGAAAGGCCGCTTTACCATTAGCCGCGATAACAGCAAAAACACCCTGTATCTGCA
GATGAACAGCCTGCGCGCGGAAGATACCGCGGTGTATTATTGCGCGGCGGTGCGC
GGCTATTTTATGCGCCTGCCGAGCAGCCATAACTTTCGCTATTGGGGCCAGGGCAC
CCTGGTGACCGTGAGCTGAGCGGCCGC (FIG. 20)
Sequence ID 19
MLGKNDPMCLVLVLLGLTALLGICQGHHHHHHDDDDKQVQLVESGGGLVQPGGSLRL
SCAASMFYPDSRCRGPSETSLGWFRQAPGQGLEAVAAIASMGGLKYYADSVKGRFTI
SRDNSKNTLYLQMNSLRAEDTAVYYCAAVRGYFMRLPSSHNFRYWGQGTLVTVS FIG.
20)
Sequence ID 20
QVQLVESGGGVVQPGKSLRLSCAASGFTFSGYGMHWVRQAPGKGLEWVALISYDES
NKYYADSVKGRFTISRDNSKNTLYLQMNSLRAEDTAVYYCAKVKFYDPTAPNDYWGQ
GTLVTVSSG FIG. 25)
Sequence ID 21
QVQLVESGGGLVQPGGSLRLSCAASMFYPDSRCRGPSETSLGWFRQAPGQGLEAVA
AIASMGGLTYYADSVKGRFTISRDNSKNTLYLQMNSLRAEDTAVYYCAAVRGYFMRLPS
SHNFRYWGQGTLVTVS (FIG. 25)
Sequence ID 22
QVQLVESGGGLVQPGGSLRLSCAASGGSEYSYSTFSLGWFRQAPGQGLEAVAAIASM
GGLTYYADSVKGRFTISRDNSKNTLYLQMNSLRAEDTAVYYCAAMFYPDSRCRGPSET
RYWGQGTLVTVS (FIG. 25)
Sequence ID 23
QVQLVESGGGLVQPGGSLRLSCAASMFYPDSRCRGPSETSLGWFRQAPGQGLEAVA
AIASMGGLTYYADSVKGRFTISRDNSKNTLYLQMNSLRAEDTAVYYCAAMFYPDSRCR
GPSETRYWGQGTLVTVS (FIG. 25)
Sequence ID 24
MHLMDWTWRVFCLLAVAPGAHS (FIG. 31)
Sequence ID 25
MKVWTFISLLFLFSSAYS (FIG. 31)

### BACKGROUND

### 1. Field of the Invention

The present disclosure provides a nanobody platform having a high affinity purification on protein A, wherein the nanobodies may incorporate diverse bioactive peptides, lipid or glycoside in its CDR1 and/or CDR3 regions. The present disclosure also provides a peptide-modified nanobody comprising non-toxic bioactive peptides and that exhibit potent anti-tumor activity. Moreover, the peptide-modified nanobody is able to be combined with other technologies, such as but not limited to bispecific antibodies and antibody-drug conjugates. Further, the present disclosure also provides production processes of the nanobodies and their use as a treatment and diagnostic agents.

### 2. Background of the Disclosure

Nanobodies (Nbs), known as single-domain antibodies derived from camelids, have emerged as a promising class of therapeutic agents in oncology. Comparing nanobodies and conventional monoclonal antibodies (MAbs) is crucial to understanding the potential and limitations of these innovative therapies in cancer treatment.

MAbs are a cornerstone of cancer treatment. Their ability to specifically target antigens expressed on tumor cells allows for targeted therapy, minimizing damage to healthy tissues. MAbs can function as receptor antagonists, as is the case with Trastuzumab, which binds to the HER2/neu receptor in some types of breast cancer, inhibiting signaling that leads to cell proliferation.

Furthermore, MAbs can recruit components of the immune system to destroy tumor cells, acting as mediators in immuno-oncology therapies. For example, ipilimumab blocks CTLA-4, an immune checkpoint, to enhance an immune response against the tumor.

Antibody-drug conjugates (ADCs) represent another innovative therapeutic avenue, where MAbs are conjugated to cytotoxic agents. The MAb acts as a vehicle that delivers the agent directly to tumor cells, allowing the use of more potent drugs with fewer systemic side effects.

Nanobodies offer several advantages compared to MAbs. Their small size confers better tumor penetration and the ability to bind to epitopes that are inaccessible to MAbs. This can translate into superior therapeutic efficacy, especially in solid tumors where penetration of larger therapeutic agents is often limited.

The production of nanobodies is also more economical as they can be expressed in prokaryotic systems like E. coli, unlike MAbs that require eukaryotic expression systems. Additionally, nanobodies are characterized by high thermal stability and the ability to refold after denaturation, an important logistical consideration for drug distribution in resource-limited regions.

However, there still exists a challenge about the purification methods of nanobodies, since many of them fail to be recognized by Protein A, a surface protein of Staphylococcus aureus used in the chromatography affinity purification technique, a widely used method for protein purification that ensures proper secondary folding selection and high purity.

In that sense, the present disclosure presents an improved nanobody, with specific modifications designed to enhance binding to Protein A resulting in high-quality peptide-modified nanobodies suitable for diverse applications for use in pharmaceuticals, diagnostics, and biotechnology. The modified nanobody of the present invention serves as the basic scaffold for the development of a versatile peptide grafting platform aimed at preserving the biological activity of the peptides while potentially enhancing their thermal and chemical stability.

Moreover, the present invention also relates to fusion proteins generated through recombinant technology, wherein two or more functional protein domains are combined into a single molecule. Traditionally, the construction of fusion proteins requires the use of linkers or additional structural elements to maintain functionality and proper folding. In contrast, the modified nanobody described herein introduces a novel one-step expression platform that enables the direct insertion of paratopes into a stable scaffold, resulting in a fully functional fusion protein without the need for linkers, post-expression modifications, or additional assembly steps. This streamlined approach not only simplifies the production process but also expands the possibilities for designing multifunctional therapeutic and diagnostic proteins with enhanced precision and efficiency.

The present invention may also be applied to bispecific antibody technologies by incorporating the peptide-modified nanobody for targeted immunotherapy. These bispecific antibodies can be engineered to include both the peptide-modified nanobody-capable of directly binding tumor-associated receptors and exerting potent antitumor effects—and an immune checkpoint inhibitor, thereby enhancing immune system activation against cancer.

For instance, a bispecific antibody combining the peptide-modified nanobody with an anti-PD-L1/PD-L2 domain could selectively bind to tumor cells, induce direct cytotoxicity, and simultaneously block immunosuppressive signaling. Alternatively, a bispecific construct containing the peptide-modified nanobody and an anti-PD-1/PD-2 domain could promote tumor targeting while increasing T cell activation and tumor clearance.

These bispecific formats are expected to demonstrate enhanced therapeutic efficacy over monotherapies and may also be used in combination with current checkpoint inhibitors such as atezolizumab, nivolumab, or pembrolizumab. Beyond PD-1/PD-L1 combinations, additional bispecific formats targeting other tumor markers or immune regulatory molecules may be developed.

Such bispecific antibodies can be constructed using recombinant DNA technologies, incorporating modifications to optimize parameters such as stability, binding affinity, and half-life. Broader configurations, including diverse antigen pairings and functional domains, may extend the therapeutic potential of these constructs to oncology and immune-mediated diseases.

The present invention further encompasses the use of the peptide-modified nanobody in Antibody-Drug Conjugates (ADCs) for targeted cancer therapy. By recognizing a tumor-associated antigen, the peptide-modified nanobody enables highly specific delivery of cytotoxic payloads to malignant cells, minimizing off-target effects. Suitable payloads may include microtubule inhibitors (e.g., MMAE, DM1), DNA-damaging agents (e.g., calicheamicin), or other novel tumor-selective compounds.

In alternative embodiments, dual-targeting ADCs may be developed by combining the peptide-modified nanobody with additional antitumor agents or immune-modulating components, thereby enhancing both specificity and therapeutic efficacy. These ADCs may be administered as monotherapies or in combination with immune checkpoint inhibitors or conventional chemotherapeutics to achieve improved clinical outcomes. The disclosed peptide-modified nanobody-based ADCs can be engineered with optimized stability, pharmacokinetic profiles, and controlled drug-release mechanisms. Furthermore, advanced configurations-such as bispecific ADCs or constructs incorporating immune-stimulatory payloads—may broaden their utility in oncology and other immune-related diseases.

The p53 tumor suppressor protein plays a critical role in maintaining cellular integrity by regulating DNA repair, cell cycle arrest, and apoptosis in response to genomic damage. As the most frequently mutated gene in human cancers, TP53 alterations often lead to loss of its tumor-suppressive function and, in many cases, the acquisition of oncogenic gain-of-function (GoF) properties that promote tumor progression, therapy resistance, and metastasis. According to mutation frequency data, p53 mutations are particularly prevalent in aggressive solid tumors, including ovarian cancer (47.27%), colorectal cancer (44.55%), stomach cancer (36.78%), pancreatic cancer (38.53%), breast cancer (26.44%), and lung cancer (40.8%). Notably, in triple-negative breast cancer (TNBC), p53 mutations are found in approximately 80% of cases, highlighting its central role in this highly aggressive and difficult-to-treat subtype. Given its high mutation rates in these malignancies, targeting mutant p53 represents a crucial therapeutic strategy for improving patient outcomes in cancers that currently lack effective targeted treatments.

Up to the moment, results shown that the mechanism of action (MoA) of the peptide-modified nanobody involves a multifaceted approach to restoring p53's tumor-suppressive functions while inhibiting its oncogenic gain-of-function (GoF) activities. This unique dual mechanism enables selective and effective elimination of cancer cells harboring p53 mutations.

One of the primary actions of the peptide-modified nanobody is the reactivation of mutant p53, which has often lost its tumor-suppressive function due to structural alterations. By binding covalently to mutant p53, specifically interacting with thiol groups via its arginine (R) and cysteine (C) residues within the RCR core sequence, the peptide-modified nanobody stabilizes mutant p53 into a wild-type-like conformation. This restored structure allows p53 to regain its transcriptional activity, enabling the expression of pro-apoptotic genes essential for tumor suppression. As a result, the peptide-modified nanobody promotes apoptosis and cell cycle arrest in tumor cells.

In addition to restoring p53 function, the peptide-modified nanobody also disrupts the gain-of-function (GoF) activity of mutant p53, which is often associated with tumor progression and resistance to therapy. A key factor in mutant p53 stabilization is phospholipase D (PLD), which plays a crucial role in maintaining pro-survival signaling. The peptide-modified nanobody directly inhibits PLD, preventing the stabilization of mutant p53 and effectively blocking its oncogenic activity. This disruption of GoF signaling leads to the reduction of tumor cell viability and suppresses pro-survival pathways that would otherwise drive tumor progression.

Furthermore, the peptide-modified nanobody induces mitochondrial permeability through modulation of VDAC1 and PPIF, leading to increased reactive oxygen species (ROS) production and the activation of ER stress. The cumulative effect of these events results in protein misfold accumulation and enhanced production of active p53, amplifying its tumor-suppressive effects. Ultimately, this series of molecular disruptions triggers caspase-independent apoptosis, mediated by key apoptotic regulators such as CDK1, HTRA2, and AIFM2. This process ensures selective tumor cell elimination while sparing normal cells, reducing the likelihood of systemic toxicity.

By simultaneously reactivating mutant p53's tumor-suppressive functions, inhibiting its oncogenic gain-of-function properties, and inducing ER stress-mediated apoptosis, the peptide-modified nanobody presents a novel and highly effective therapeutic strategy against cancers driven by p53 mutations. Based on current knowledge, the molecule is understood to act through this mechanism; however, its activity is not limited to this MoA.

The present disclosure is the first to recombinantly incorporate a bioactive peptide and its derivatives in a single-domain antigen-binding fragment of humanized camelid heavy-chain antibody (nanobody), presenting a suitable alternative for the sole or complementary treatment of patients with aggressive tumors.

Moreover, the nanobody itself presented herein has specific modifications that improve its affinity to protein A, facilitating its purification and allowing its production in eukaryotic cells. The improved nanobody can serve as a stable platform for the precise integration of other sequences, besides 3-Nantc, as our studies conclude that CDR1 and/or CDR3 of the nanobody per se would accommodate any bioactive peptide, lipid or glycoside with various pharmacological properties, such as but not limited to analgesic, antibiotic, anti-inflammatory, anti-viral, anti-cancer, among others.

The production process of the modified nanobody itself and the peptide-modified nanobody are also presented herein.

### SUMMARY

The present disclosure provides a nanobody platform comprising a single-domain antigen-binding fragment of humanized camelid heavy-chain antibody having a protein A binding site.

Preferably, the nanobody platform comprises at least 70% of similarity with the SEQ ID 1.

More preferably, the nanobody platform comprises at least 70% of similarity with the SEQ ID 1 and, additionally, has at least one of the following features: i) an interval between the S25 and S26 amino acids for bioactive peptide insertion; ii) an interval between the A91 and R92 amino acids for bioactive peptide insertion; and/or iii) the presence of the K amino acid in the 51 position.

The size and amino acid composition of the interval will be dependent on the size and the composition of the bioactive peptide to be inserted therein. The size and amino acid composition of the interval will be dependent on the size and the composition of the bioactive peptide to be inserted therein. The bioactive peptide includes peptides, lipides, glycosides or mixtures of them.

The present disclosure provides a peptide-modified nanobody incorporating a bioactive peptide incorporated in the CDR1 region located between the S25 and S26 of the SEQ ID 1.

The present disclosure provides a peptide-modified nanobody incorporating a bioactive peptide incorporated in the CDR3 region located between the A91 and R92 of the SEQ ID 1.

The present disclosure provides a peptide-modified nanobody incorporating a bioactive peptide incorporated in the CDR1 and/or CDR3 region of the nanobody as defined above.

The present disclosure provides a peptide-modified nanobody incorporating a non-toxic bioactive peptide used for the treatment of aggressive solid and non-solid tumors.

More specifically, the bioactive peptide incorporated in the peptide-modified nanobody is selected from the group comprising the SEQ ID 2, SEQ ID 3, SEQ ID 4, SEQ ID 5, SEQ ID 6, SEQ ID 7 and/or SEQ ID 8.

The bioactive peptide could also be selected from the group comprising at least 70% of similarity with the bioactive peptides SEQ ID 2, SEQ ID 3, SEQ ID 4, SEQ ID 5, SEQ ID 6, SEQ ID 7 and/or SEQ ID 8.

Specifically, the peptide-modified nanobody having the bioactive peptide is therapeutically effective for the treatment of aggressive solid tumors, such as breast, lung, prostate, colon, skin, brain, pancreas, or kidney cancer. More specifically, it is used for the treatment of triple negative breast cancer patients.

In a different aspect, the present disclosure provides a recombinant production process of the nanobody or the peptide-modified nanobody, comprising the following steps:
a) Construction of systems for expression control and amplification;
b) Purification and eukaryotic cells transformation; and
c) Clone selection, nanobody expression and purification.

In an embodiment, the peptide-modified nanobody comprises a non-toxic bioactive snake venom peptide.

Use of the nanobody platform or peptide-modified nanobody for the treatment, prevention or diagnosis of patients is also disclosed herein.

### BRIEF DESCRIPTION OF THE DRAWINGS

FIG. 1 shows a representation of a peptide engrafted into the different regions of the nanobody platform (gray): in CDR1 (A), in CDR3 (B), and in both CDR 1 and 3 (C) regions.

FIG. 2 shows the amino acid sequence for the peptide-modified nanobody snake venom bioactive peptide (bold) inserted into the nanobody scaffold, referred to herein as the peptide-modified nanobody.

FIG. 3 presents the MS/MS characterization that confirmed the peptide-modified nanobody sequence through the m/z relation. The peptide represents an exclusive sequence from the variable region of human immunoglobulin, corresponding to the positions 95 to 107 in the nanobody sequence. The b-series (blue) and y-series (red) of ions combined cover 100% of the peptide sequence as indicated in the right-hand table. Below, the sequence is spot showing 96.9% of coverage for amino acids residues and 96.7% coverage for mass.

FIG. 4 illustrates the untreated triple negative tumor cells (MDA-MB-231) vs. treated with the peptide-modified nanobody. Treated tumor cells are presented in lower numbers. No difference in cell number was noted for the benign cell line (HB4a). MDA-MB-231 cells incubated in the presence and absence of the peptide-modified nanobody (20 μg/mL) for 72 hours. Untreated (0 μg/mL) triple negative tumor cells exhibited a larger number of cells. Treated (20 μg/mL) benign cells showed a minor difference in number of cells.

FIG. 5 shows the evaluation of the peptide-modified nanobody on cellular viability against triple negative breast cancer (TNBC) cells and mammary benign cells. MDA-MB-231 (green line) and HB4a cells (blue line) were treated for 72 h at the concentration range of 4-20 μg/mL. Cellular viability assay was evaluated using the MTT method. Data are shown as the mean±SEM of a triplicate. Significant differences between control (0 μg/mL) and treated cells are designated as * p<0.05, ** p<0.01, and **** p<0.0001, according to two-way ANOVA and a Dunnett post hoc test.

FIG. 6 shows the DNA and Protein sequences wherein the Kozak sequence to enhance the DNA transcription is in pink, the signal peptide to direct the protein to cytosol after intracellular production is in blue and the Stop translation codon is in red.

FIG. 7: plasmid linearization check. M: GeneRuler 1 kb DNA Ladder; 1) non-linearized reference plasmid pCHOm_3.1; 2) linearized reference plasmid pCHOm_3.1; 3) non-linearized reference plasmid pCHOm_3.1—applied as a positive control of the reaction; 4) linearized reference plasmid pCHOm_3.1—applied as a positive control of the reaction; 5) non-linearized work plasmid pCHOm_3.1_nanobody; 6) linearized work plasmid pCHOm_3.1_nanobody. DNA linearization was completed and the plasmid was ready for the next preparation phase.

FIG. 8: Expression test growth curve. A) X axis: Time in days; primary Y axis: cell concentration; secondary Y axis: viability expressed in %. In orange: Viability curve; In blue: cell concentration curve B) X axis: Time in days; primary Y axis: LN of cell concentration; secondary Y axis: viability expressed in %. In orange: Viability curve; In blue: LN of cell concentration curve.

FIG. 9: 5L-Bioreactor production growth curve. A) X axis: Time in days; primary Y axis: cell concentration; secondary Y axis: viability expressed in %. In orange: Viability curve; In blue: cell concentration curve B) X axis: Time in days; primary Y axis: LN of cell concentration; secondary Y axis: viability expressed in %. In orange: Viability curve; In blue: LN of cell concentration curve.

FIG. 10: PrismA Chromatography from Cultivation supernatant. The red arrow shows the elution peak of the nanobody.

FIG. 11: SDS-PAGE of Nanobody purification by ProteinA Chromatography. 1—Molecular weight marker. 2—Filtrated supernantant. 3—Chromatography flowthrough 1. 4—Chromatography flowthrough 2. 5—Chromatography flowthrough 3. 6—Chromatography Elution peak. Red arrow shows the Nanobody band.

FIG. 12: Western Blotting of Eluted fraction from Protein A Chromatography. 1—Eluted fraction. 2—VHH standard.

FIG. 13—PrismA Chromatography from Cultivation supernatant. The red arrow shows the elution peak of nanobody.

FIG. 14: Rapid A Chromatography from Cultivation supernatant. The red arrow shows the elution peak of nanobody.

FIG. 15: SDS-PAGE of Nanobody purification by ProteinA Chromatography and Membrane Chromatography. 1: Molecular weight marker. 2: Filtrated supernatant. 3: Chromatography flowthrough. 4: PrismA Elution. 5: PrismA Ultrafiltration retentate. 6: PrismA Ultrafiltration filtrate. 7: Sterile PrismA final product. 8-9: Sartobind flowthrough. 10: Sartobind elution. 11: Sartobind Ultrafiltration retentate.

FIG. 16: DNA and Protein sequences applied to E. coli expression. In pink: Restriction enzyme sites; In green: His-tag sequence. In red: Stop translation codon.

FIG. 17—Anion Exchange Chromatography from Refolding.

FIG. 18 (A-B)—SDS-PAGE of Anion Exchange Chromatography Q65. 1: Molecular marker (Mark12, Thermo lot 2629204). 2: Solubilized supernatant. 3: Solubilized pellet. 4: Solubilized filtrated. 5: Refolding pellet. 6: Refolding supernatant. 7: Refolding filtrate. 8: Q65 Flowthrough F2. 9: Q65 Flowthrough F3. 10: Q65 Flowthrough F4. 11: Q65 Elution F6. 12: Q65 Elution F7 13: Q65 Elution F8. 14: Q65 Elution F9. 15: Q65 Elution F11. 16: Q65 Elution 12. 17: Q65 Elution F14. 18: Q65 Elution F15. 19: Q65 Elution F16. 20: Q65 Elution F17. 21: Q65 Elution F18. 22: Q65 Elution F19. 23: Q65 Elution F6 concentrated 3×. 24: Q65 Elution F7 concentrated 3×. 25: Q65 Elution F8 concentrated 3×. 26: Q65 Elution F9 concentrated 3×. 27: Q65 Elution F15 concentrated 3×. 28: Q65 Elution F17 concentrated 3×. 29: CIP Q65. Red lines represent nanobody region.

FIG. 19—SDS-PAGE of Q65 Sterile Concentrated Flowthrough. 1: Molecular marker (BLUEeye, Sigma lot BCCJ8209). 2: Q65 Sterile Concentrated Flowthrough. 3: Q65 Sterile Concentrated Flowthrough concentrated 3×. Red lines represents nanobody region.

FIG. 20: DNA and Protein sequences applied to P pastoris expression system. In pink: Restriction enzyme sites; In blue: signal peptide to direct the protein to cytosol after intracellular production; In green: His-tag sequence. In red: Stop translation codon.

FIG. 21: Expression profile of clones: SDS-PAGE from screening cultures M: Molecular marker BLUeye PPM (Sigma—Lot: BCCJ8209) 1-10: supernatant of cultivation from different clones; 11: Negative control—no transformed P pastoris.

FIG. 22: PrismA and IMAC Chromatography from Cultivation supernatant. The green rectangles shows the elution peaks of nanobody. A: Clone 30. B: Clone 21. C: Clone 31. D: Clone 35.

FIG. 23: SDS-PAGE from IMAC eluates concentration and final product sterile filtrate. 1: Molecular marker BLUeye PPM (Sigma—Lot: BCCJ8209). 2-5: Concentrated IMAC elution clones 30, 21, 31 and 35. 6-9: Sterile filtrated IMAC final product clones 30, 21, 31 and 35. Red line indicates de nanobody region.

FIG. 24: SDS-PAGE from ProteinA eluates concentration and final product sterile filtrate. 1: Molecular marker BLUeye PPM (Sigma—Lot: BCCJ8209). 2-4: Concentrated ProteinA elution clones 21, 31 and 35. 5-7: Sterile filtrated ProteinA final product clones 21, 31 and 35. 8-10: Sterile filtrated ProteinA final product clones 21, 31 and 35 concentrated 3×. Red line indicates de nanobody region.

FIG. 25—Comparison of the sequences and structures of the NanoPeps with the heavy chain of a model antibody (VH3) for interaction with ProteinA. (A) crystallographic structure of the VH3/ProteinA complex obtained from the Protein Data Bank with access code 1DEE (left) and highlight of VH3 interface and hotspot residues for protein A binding (right). (B) Alignment matrix constructed for VH3 and NanoPeps sequences. The hotspot residues for ProteinA binding are underlined in green and the substitution of lysine K for threonine in Nanopeps is highlighted in red. (C) NanoPeps structures with indication of the respective hotspot residues for ProteinA binding determined from the comparison with VH3.

FIG. 26—Comparison of the ProteinA binding interfaces of VH3 and NanoPeps. The structural diagrams are shown as cartoon for the structures of VH3 (gray) and NanoPeps (salmon) with emphasis on the positions of the hotspots (green). The superimpositions of the NanoPeps were carried out using the Ca carbon atoms of the VH3 hotspots as reference structure.

FIG. 27—Evaluation of native NanoPeps-ProteinA complexes dymanics by molecular dynamics simulations. The structures of model complexes between ProteinA (orange) and NanoPeps (salmon) are showed as cartoon diagram for initial systems (A) and final structure at 100 ns of molecular dynamics (B). The peptide fragments are highlighted in cyan.

FIG. 28—Evaluation of mutant NanoPeps-ProteinA complexes dymanics by molecular dynamics simulations. The structures of model complexes between ProteinA (orange) and NanoPeps (salmon) are showed as cartoon diagram for initial systems (A) and final structure at 100 ns of molecular dynamics (B). The peptide fragments are highlighted in cyan.

FIG. 29—Evaluation of binding affinity of NanoPeps to ProteinA. Density plot for binding affinity estimates of complex formation for VH3 and NanoPeps (native and mutants) with ProteinA employing MM/GBSA method. The mean binding affinities for each system are indicated as vertical lines inside de graphs and are showed in Table 1.

FIG. 30—Comparison of efficacy between two tested compounds obtained in two different expressions systems (E. coli vs. CHO).

FIG. 31—Percentage of viable cells obtained in 72 hrs MBAMD231 for a compound with different signal peptides, from albumin and IgG. Although same activity is observed, there are great differences in productivity (titer).

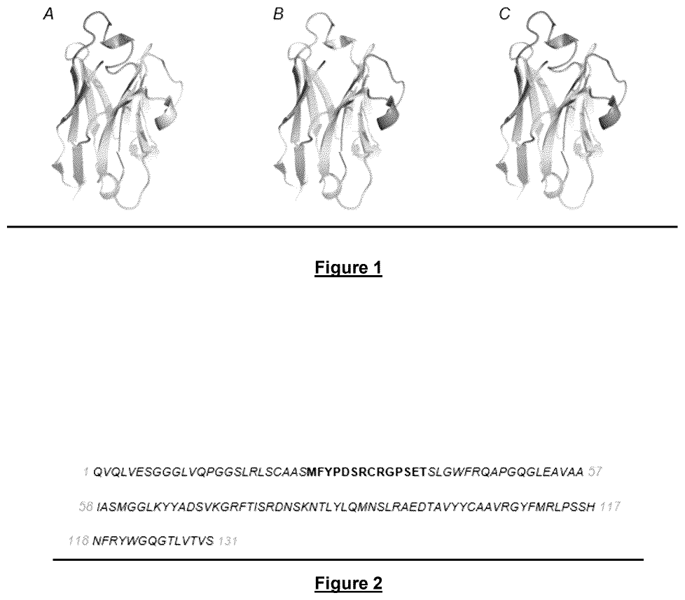

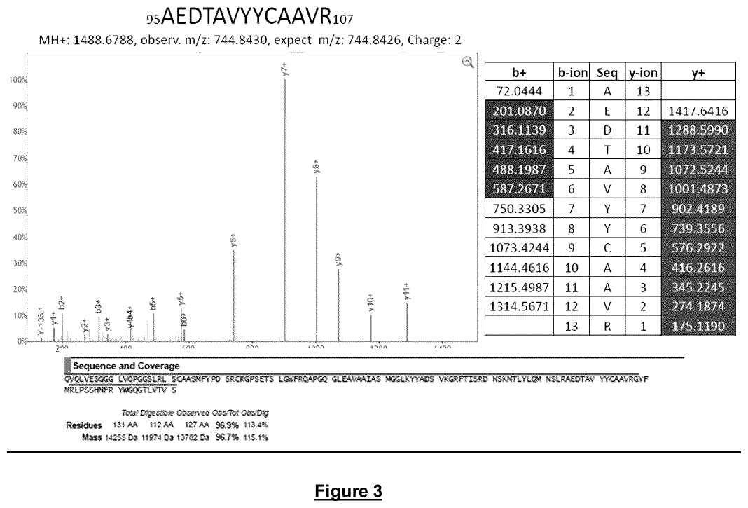

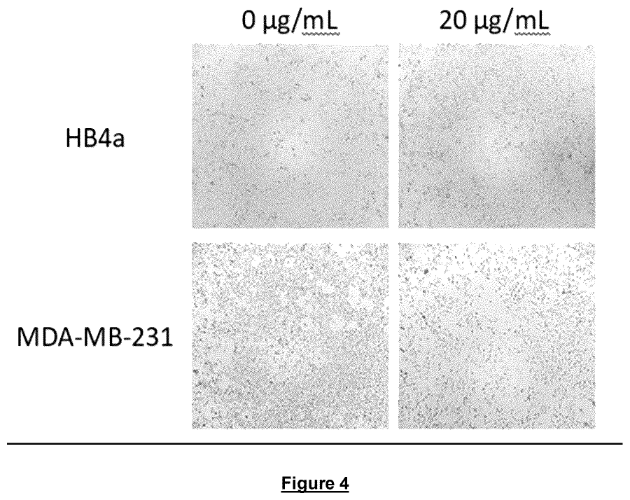

### DETAILED DESCRIPTION OF THE DISCLOSURE

The terms “peptide-modified nanobody”, “nanobody” and “Nanopep” are used interchangeably herein and comprise a single-domain antigen-binding fragment of humanized camelid heavy-chain antibody having a protein A binding site. The nanobody preferably comprises at least 70% of similarity to the SEQ ID 1, as defined herein. Traditional purification methods of nanobodies without a His tag require optimization for each nanobody, making the introduction of a Protein A binding site essential for generic purification of nanobodies from microbial and eukaryotic systems. Affinity chromatography using Protein A resins, which select properly folded human proteins, ensures higher purity and is an industry standard for monoclonal antibodies (mAbs) as well. With this information in mind, the peptide-modified nanobody would be characterized by a purity preferably equal or greater than 80%, more preferably, a purity equal or greater than 85%, 90%, 95% or 99%. The protein A binding site herein is a set of amino acids located upstream and downstream the S25, S26, A91, R92 or K51 amino acids of the SEQ ID 1 or its similar sequences. Preferably, the protein A binding site is a region comprised by a set of 10 amino acids, more preferably, the region is a set of 9, 8, 7, 6 or 5 amino acids, located upstream or downstream of any of the S25, S26, A91, R92, K51 amino acids of the SEQ ID 1 or its similar sequences. The nanobody can incorporate at least one bioactive peptide incorporated in at least one complementarity-determining regions (CDRs). The bioactive peptide includes peptides, lipides, glycosides or mixtures of them. As an example, the CDR1 region that can incorporate a peptide is located between the amino acids S25 and S26 of the SEQ ID 1. As another example, the CDR3 region that can incorporate a peptide is located between the amino acids A91 and R92 of the SEQ ID 1.

The nanobody sequence of the present disclosure can be used as a platform that includes one or more peptides in the CDR1 and/or CDR3 regions.

The nanobody of the present disclosure can be produced using eukaryotic cells as, for example, but not limited to, Chinese Hamster Ovary (CHO) cells, bacterias as, for example, but not limited to Escherichia colli (E. coli) and/or yeasts as Pichia pastoris (P pastoris). In a preferred manner, the nanobody of the present disclosure can be obtained through specific modifications in the amino acids of the h-NbBcII10FGLA sequence (PDB code: 3EAK) to facilitate the recombinant production in eukaryotic systems and improve its purification efficiency. One example of peptide-modified nanobody can be seen with the incorporation of the non-toxic bioactive snake peptide in the nanobody structure, as described below.

The term “Protein A” is used herein to define a protein derived from a strain of Staphylococcus aureus that contains five regions that bind to the Fc region of IgG and is used as an affinity ligand during the nanobody purification. Both native protein A (nProtein A) and recombinant protein A (rProtein A) ligands are included herein. Besides the well-known affinity for the Fc region of IgG, protein A also has affinity for certain variants of the Fab region, and consequently, protein A chromatography media can be used for the purification of Fab and F(ab′)2 fragments.

The term “bioactive peptide(s)” includes one or more peptides, lipides, glycosides or mixtures of them that can be incorporated inside the nanobody molecule, wherein the nanobody and the bioactive peptide becomes a unique molecule. The term “bioactive snake peptide(s)” or “bioactive snake venom peptide(s)” or “non-toxic bioactive snake peptide(s)” comprises the C-terminal part of the crotoxin protein obtained from Crotalus durissus terrificus and its derivatives. Specifically, the peptide-modified nanobody shows a purity equal or greater than 80%, more preferably, a purity equal or greater than 85%.

The bioactive peptide(s) of the present disclosure are active against tumor cells, more specifically, against TNBC cells. The bioactive peptide(s) comprises the SEQ ID 2 (also called herein “3-Nantc”) and one or more of its derivatives (SEQ ID 3, SEQ ID 4, SEQ ID 5, SEQ ID 6, SEQ ID 7, SEQ ID 8). In a more preferable manner, the bioactive peptide has at least more than 70%, or more than 80%, or more than 90%, or 100% similarity with SEQ ID 2, SEQ ID 3, SEQ ID 4, SEQ ID 5, SEQ ID 6, SEQ ID 7 or SEQ ID 8. A peptide-modified nanobody comprising the bioactive peptide means that the peptide can be incorporated in the CDR1 and/or CDR3 region of the nanobody. Preferably, the bioactive peptide is incorporated in the CDR1 region.

The term “modulation of tumoral cells” comprises the modulation (increasing, decreasing or maintenance) of at least one of the following parameters in tumoral cells when using the peptide-modified nanobody having bioactive peptide(s): cellular proliferation, apoptosis, necrosis and the cycle progression when using the peptide-modified nanobody with the bioactive peptide(s). The modulation of tumoral cells is also dependent on the modulation of benign cells, preferably it is expected that the bioactive peptide(s) are, simultaneously, active against the growth or survival of tumoral cells while having no or few effects over benign cells. In a preferable manner, the modulation of tumoral cells using the peptide-modified nanobody decreases the viability of cancer cells while not decreasing the viability of benign skin cells. Moreover, the peptide-modified nanobody preferably decreases the proliferation rate of tumoral cells. The peptide-modified nanobody preferably causes a marked difficulty of tumoral cells to proceed to G0/G1 stage, while it leads to an arrest in both S and G2/M phases. The peptide-modified nanobody preferably induces apoptosis in tumor cells.

The term “therapeutically effective” or “therapeutical agent” comprises the use of the peptide-modified nanobody as a sole therapy or in combination with or in addition to other therapies for the modulation of one or more aspects of tumoral cells, while preserving the benign cells in a cancer patient. It can preferably be used in a solid or non-solid tumor cancer of a patient. For example, when incorporating the bioactive snake peptide, it can be used in a patient against a solid tumor breast cancer, even more preferably a solid tumor triple-negative breast cancer. Also, the peptide-modified nanobody having bioactive peptide(s) may be administered in vivo or in vitro. The peptide-modified nanobody with bioactive peptide(s) of the present disclosure is effectively capable of being used in combination with a pharmaceutical composition, including at least one pharmaceutically acceptable vehicle as a carrier, a diluent and/or an excipient. It also can be used for preventing or diagnosing diseases, the disease(s) being defined in accordance with the peptide incorporated in the nanobody and its properties.

The protein synthesis of the nanobody itself or the peptide-modified nanobody is performed through recombinant expression, which comprises at least one of the following steps: a) construction of systems for expression control and amplification; b) purification and eukaryotic cells transformation; and c) clone selection, nanobody expression and purification. In a preferred embodiment, the synthesis of the nanobody or the peptide-modified nanobody comprises at least one of the following steps: a) constructing a plasmid vector; b) amplifying the vector in E. coli; c) purifying the plasmid material; d) transfecting eukaryotic cells; e) selecting the clone of interest; f) expressing the protein of interest; g) purifying the protein of interest. The present invention further encompasses the recombinant production of modified nanobodies using plasmids as expression vectors. However, other expression systems may also be employed to optimize yield, stability, and functionality. Alternative vectors include viral vectors (such as lentiviral or adenoviral vectors) for efficient gene delivery, bacterial artificial chromosomes (BACs) for large-scale protein expression, and yeast or mammalian-based expression systems utilizing episomal vectors. These alternative approaches enable flexible production strategies, allowing adaptation to different host cells, scalability requirements, and therapeutic applications.

The present disclosure is illustrated below by reference to the following examples. However, one skilled in the art will appreciate that specific methods and results discussed are merely illustrative of the invention, as innumerable variations, modifications, applications, and extensions of these embodiments and principles can be made by one skilled in the art without departing from the spirit and scope of the disclosure.

### EXAMPLES

### Example 1—CHO Cell Line Development and Results

### 1.1. Construction of Expression Plasmids (in Silico)

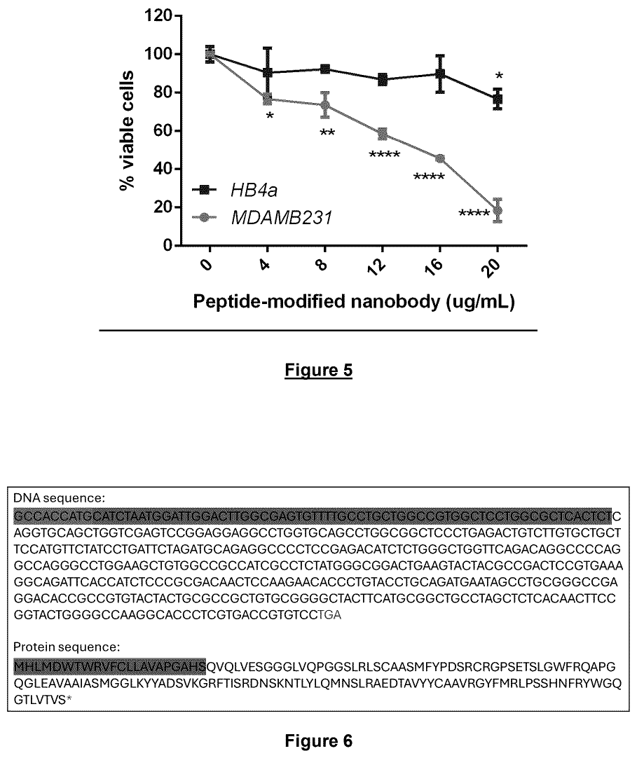

Specifically concerning the construction for CHO cells, the inventors added a signal peptide sequence (MDWTWRVFCLLAVAPGAHS—SEQ ID 9) preceded by the Kozak sequence (gccaccatg). The Eco-RV restriction site was chosen for the insertion of the DNA of interest into the pCHOm 3.1 vector. The synthesized sequence underwent codon optimization (Cricetulus griseus) avoiding the Nru-I restriction site, which was used for the subsequent plasmid linearization. pCHOm 3.1 vector has kanamycin resistance gene (aph) for transformed E. coli selection; PGK (phosphoglycerate kinase) promoter for cell constitutive recombinant protein expression and DHFR (dihydrofolate reductase) gene to the Methotrexate (MTX) selection (see, Gibco—MAN0025765 for more vector info). Gene synthesis and cloning services into the expression vector were performed by Genscript. The material was shipped lyophilized, with 4 μg of DNA. Additionally, Genscript provided a quality control report with sequencing data of the synthesized DNA to confirm the correct synthesis and cloning of the molecule. The Genscript package of services provides a proprietary algorithm to codon optimization. The output of the software was the synthesized sequence. FIG. 6 shows the sequence of DNA suggested by Genscript and the in silico translation. An alignment between PHP and the synthesized sequences was done to compare if the amino acid sequence was maintained the same (not shown). Therefore, it was possible to confirm that the Genscript service was done properly.

### 1.2. DNA Preparation for Transfection

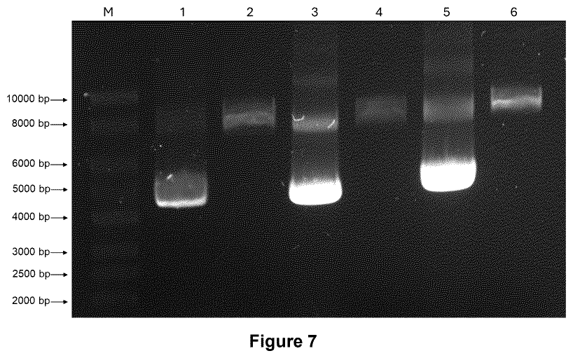

With the plasmids in hand, the inventors conducted procedures for replicating the material to achieve the necessary quantity for transfections; linearization to increase the recombination and ensure efficiency of the integration of the insert to cell genome; and purification to enhance the chances of success in the preparation, as described in detail below. The DNA preparation step was divided into four parts, which were, E. coli transformation; plasmid DNA extraction; plasmid linearization and DNA purification. E. coli transformation was successful once the colony count was more than 100 colonies per Petri dish. Plasmid DNA extraction yields around 25 μg of DNA per preparation. Therefore, there were necessary five preparations to obtain the desired amount of DNA, considering an expected material loss at DNA purification step. All preparations passed on “280/260” ratio quality test. Linearization reaction was checked by agarose electrophoresis. FIG. 7 shows the DNA after completing linearization reaction. Quantification after purification step showed a yield of 90 μg of total pCHOm_3.1_nanobody. Purified and linearized DNA solution passed on “280/260” ratio quality test and was considered ready for cell transfection.

1.2.1. Transformation in E. coli

The initial step in preparing the DNA used involved transforming E. coli bacteria, capable of replicating the plasmid. “One Shot™ TOP10 Chemically Competent E. coli” cells (26077075B) were used. Transformations were performed using a heat shock method according to the manufacturer's recommendations (Invitrogen—MAN0000633). Equipment used in these steps were a biosafety cabin (PACHANE—Model PA400); and a micropipette set and water bath (CapLab—model SSD 5L). The transformed suspension was inoculated onto Petri dishes prepared with LB agar medium (Lot 019/23—FOR-MS.031) supplemented with kanamycin sulfate (Lot 2556471). Incubation was carried out at 37° C. for 18 hours in bacterial Incubator (SPLABOR—SP 101/30.1).

### 1.2.2. Plasmid DNA Extraction

Following successful bacterial transformations, the next step was to extract plasmid DNA in sufficient quantity for subsequent stages. For this purpose, the “PureYield Plasmid Midiprep System” kit from Promega (Lot 587624) was used as per the manufacturer's recommendations (Promega—TM253). The extracted material was quantified using spectrophotometry (λ=260 nm) with a Tecan instrument, using the Nanoquant tool (Infinite Pro 200). The equipment reads at A=260 nm, and the “260/280” ratio was used as a quality parameter for the solution. Extractions were deemed acceptable for “260/280” ratios greater than or equal to 1.8. With the material in adequate quantity and quality, the next step was plasmid linearization. The enzyme Nru-I (New England Biolabs) was used according to the manufacturer's recommendations. Quality analysis of the linearized material was performed by agarose gel electrophoresis at 0.8% (Fastbio Tablets—Lot D0057). 1 kb DNA LadderGene Ruler (LOT SLCK0031) was used as the molecular weight standard. Electrophoresis was carried out with the equipment from KASVI (model K3316H).

### 1.2.3. DNA Purification

Upon successful confirmation of the linearization reaction, the material underwent a DNA precipitation-based purification process. For this, 1/10 volume of 3 M sodium acetate solution (Dinâmica—Lot 111148) was added to each tube containing the linearization product, followed by gentle homogenization. Subsequently, 2.5 times the volume of 96% ethanol (Sigma—Lot 1274427308) was added, followed by homogenization and incubation at −20° C. in the freezer (Consul—model CRD37E) for approximately 18 hours. After this period, the material was centrifuged at 12,000 g, 4° C. for 45 minutes. The pellet was then washed twice with 70% ethanol. After the second wash, the pellet was incubated at 37° C. to completely evaporate the ethanol. Finally, the pellet was resuspended in ultrapure water. The material was quantified again, as described in section 1.2.2 (where is this?), following the same quality criteria.

### 1.3. Cell Line Development

The cell line development is based on the transfection and subsequent selection of cells that can grow in the selective culture medium. It starts with non-transfected cells cultivation, effective event of transfection and systematic use of selective molecules to enrich the cell pools with the recombinant ones. To be successful, it is important to respect the cell growth cycle and the timing of the transfection.

### 1.3.1. Cell Bank Thaw and Cultivation Conditions

The ExpiCHO-S™ Cells (cGMP-banked) (Lot 2492989) were stored in liquid nitrogen. This cell bank thaw was carried out as described by manufacturer s manual: The vial number 5 was thawed by incubation in 37° C. water bath (CapLab—model SSD 5L) for 2 minutes and then the entire contents of the cryovial was transferred into a 125-mL polycarbonate, disposable, sterile, vent-cap Erlenmeyer shaker flask containing 30 mL of ExpiCHO™ Expression Medium (Lot: 2492989) pre-warmed to 37° C. All the manipulations were done in a biosafety cabinet (SPLABOR—model SP-SBIIA1-960/4). The ExpiCHO-S™ Cells (cGMP-banked) (Lot 2492989) were stored in liquid nitrogen. This cell bank thaw was carried out as described by manufacturer's manual: The vial number 5 was thawed by incubation in 37° C. water bath (CapLab—model SSD 5L) for 2 minutes and then the entire contents of the cryovial was transferred into a 125-mL polycarbonate, disposable, sterile, vent-cap Erlenmeyer shaker flask containing 30 mL of ExpiCHO™ Expression Medium (Lot: 2492989) pre-warmed to 37° C. All the manipulations were done in a biosafety cabinet (SPLABOR—model SP-SBIIA1-960/4). The cells were incubated in a shaker incubator (Infors-HT model Minitron) with 80% relative humidity, 37° C., and 5-8% CO2 on an orbital shaker platform of 125±5 rpm. A sample was taken to evaluate the viable cell density and cell viability. The trypan blue reagent (Lot 2785344) was used for differentiation between viable and non-viable cells by microscopy. The microscope Kasvi, model K551VT, was used in all the analysis. As a quality control of this step, cell viability must be higher or equal to 90%. The expression system manual suggested that the log growth phase is granted until the cell density is under 2×10⁷ viable cells/mL. Therefore, passages were done when the cultivations were between 2×10⁶ and 8×10⁶ viable cells/mL. Seeding passages were done at the cell's concentration between 1.5×10⁵ and 2×10⁵ cells/mL. After 3 passages, an internal research cell bank was prepared, combining the expression system manual suggestions (Cell bank code: BC003/23). For that, the cultivation was ready for cryopreservation when cell's concentration was between 4×10⁶ and 6×10⁶ cell/mL and viability higher or equal to 95% before harvest. Cultures were harvested by centrifuging 200 g for 5 minutes (Centrifuge ANCO model CDR16000-220V). Pelleted cells were resuspended at a final density of 1×107 viable cells/mL in 90% fresh ExpiCHO™ Stable Production Medium (Lot 006/24 FOR-MS.072) and 10% DMSO (Lot BCCG0331). Aliquots of 1 mL were done and the cryovials were frozen at the rate of 1° C. per minute (Mr Frosty™ freezer container) until reach −80° C. in ultrafreezer (ColdLab—model: CL 200-80 Freezer Vertical). The cell bank was stored in liquid nitrogen tank (Volta—model 47SQ) for long-term storage.

### 1.3.2. Cell Transfection

The transfection method is a lipid-based transfection using ExpiFectamine™ CHO Reagent (Lipofectamine) (Lot 2532080). It starts one day before the transfection event (day −1). A split of the cultivation was carried out to start with 5×10⁵ viable cells/mL. At the day zero, cell density should be between 1.0×10⁶ and 1.5×10⁶ viable cell/mL and the viability between 95% and 99%. The volume was adjusted to 30 mL and 1×10⁶ viable cell/mL. 40 μL of Cold ExpiFectamine™ CHO Reagent (Lot 2532080) was mixed with 0.96 mL of OptiPRO™ Serum Free Medium. (Lot 2451723). In parallel, 60 μg of purified DNA was mixed to OptiPRO™ Serum Free Medium (Lot 2451723) to a final volume of 1 mL. The previously prepared solutions were mixed and carefully added to the cell flask. The flask was cultivated as described at section 1.3.1. 48 hours later, the cell's density and viability were checked, and the cultures were put into selective conditions.

### 1.3.3. Selections

At this point, there is a pool of transfected and non-transfected cells in the culture flask. As the pCHOm 3.1 plasmid was used in this stage, it is possible to use the Methotrexate (MTX) molecule as a selective agent. This selection phase is divided into two phases: selection 1 and selection 2. The main difference between the phases is the MTX concentrations in the cultivations. For this, the transfection cultivation was split in two flasks with fresh ExpiCHO™ Stable Production Medium (Lot 006/24 FOR-MS.072) with 100 nM and 200 nM of MTX (Lot 001/24—FOR-MS.071), respectively. Viability loss is expected as the non-transfected cells are no longer able to grow in this new environment. As cultivation time goes by, the transfected cells grow, and the cell viability of cultivation rises again. The target cell viability is 85%. At this point, a new passage is done with each cultivation, giving rise to four cultivations in total. Two of the cultivations (one from 100 nM selection and the other from 200 nM selection) with 500 nM of MTX and the other two cultivations (one from 100 nM selection and the other from 200 nM selection) with 1000 nM of MTX. The target cell viability for selection 2 is 90%. All the passages started with 3×10⁵ viable cell/mL with fresh ExpiCHO™ Stable Production Medium (Gibco Lot 2665884) supplemented with 4 mM of GlutaMax (Lot 2523109). Backup cryopreserved vials were made after each selection phase, following the procedures reported in section 4.3.2. Furthermore, expression tests were done after the selection 1 and 2 to detect a desired protein production.

### 1.3.4. Expression Test

Expression tests were carried out by seeding fully recovered cell pools (viability >90%) at 3×10⁵ viable cells/mL using 120 mL ExpiCHO™ Stable Production Medium without selective pressure in 500-mL shake flasks. Cultures were incubated at 37° C., 80% relative humidity and 5-8% of CO₂. Cultures were sampled on days 0, 4, 7 and 8 to determine the cell density, viability, and productivity until culture viability dropped below 50% or reached 14 days. The culture was fed at the days 4, 7 and 8 with glucose (Glucose solution—Lot 2708586) final concentration of 4 g/L. The cultivations were purified as described below. Productivity was detected by SDS-PAGE.

As described in methodology after cell thaw, passages were done every time the cultivation reached 1.0×10⁶ to 8.0×10⁶ viable cell/mL. The start concentration after the passage was between 1.5×10⁵ and 4.0×10⁵. Quality control of viability higher than 90% was followed. The internal research bank was produced after three passages of first cell thaw. Starting from work cell bank, four more passages were enough to reach the transfection requirement. The cultivations viability at the transfection time were between 91.34 and 100%.

After three days of transfection event, the cultivations were with viability ranging between 87.5 and 93%. The drop of viability is expected as the selective molecule makes pressure to non-transfected cells die and the cultivation gets enriched of transformed cells. At the time of the transfection's subsequent passage—after 10 to 13 days—the cultivations were split. One part was used in the selection 2 initiation and the other was used to start an expression test.

Expression test was carried out starting a cultivation with around 3×10⁵ viable cell/mL in a supplemented culture medium. Four, seven and ten days after the start, cultivation was supplemented with 4 g/L of glucose. Quality control of viability higher than 90% was followed.

Growth curve showed exponential growth until day 4. Cultivation started a maintenance phase that could be detected on day 5 and, after day 7, it is possible to see a death phase. Cell viability started to drop after day 7 also. Maximum cell density was reached at day 6. A possible explanation of this cultivation behavior is a lack of nutrient, especially after day 4. Perhaps, glucose supplementation is not enough in 4 g/L or it is applied late in the cultivation. Other nutrients could be growth limiters, e.g. glutamine. More studies are necessary to improve the growth performance and, consequently, reach higher protein titters.

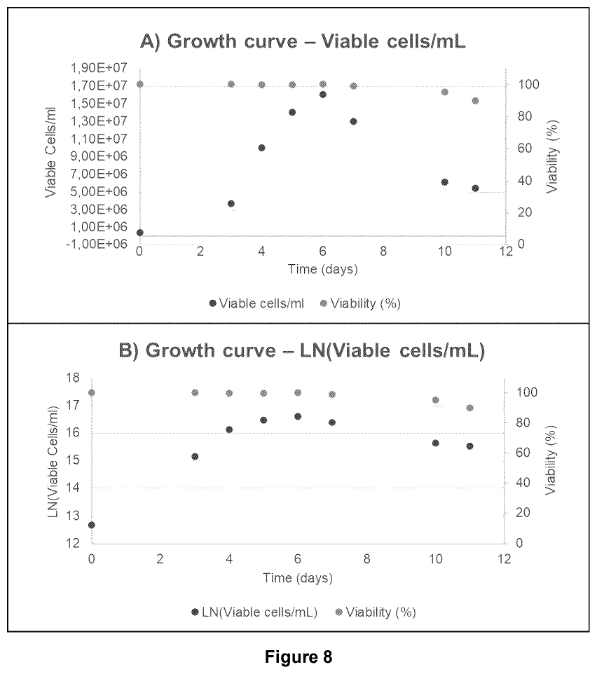

An SDS-PAGE was carried out to detect protein expression after 5 and 6 days of cultivation starting (FIG. 8). SDS-PAGE analysis shows that the cultivation started from selection 1 phase can potentially express Pepcrotament protein. The production confirmation and bioprocess yield were confirmed after protein purification steps.

### 1.3.5. Biorreactor Production

Bioreactor production was carried out by seeding fully recovered cell pools (viability >90%) at 8.5×10⁵ viable cells/mL in a stirred tank with 3300 mL of mL ExpiCHO™ Stable Production Medium (Gibco Lot 2665884) supplemented with 4 mM of GlutaMax (Lot 2523109), without selective pressure. The bioreactor used was InforsHT—model Minifors 2 with 4 liters glass vessel. The impeller applied was pitched blade. Cultivation conditions were maintained as follows:

| | | |
|---|---|---|
| Parameter | Setpoint | Control |

| pH | 7.10 | | Cascade (NaOH; CO₂) |
|---|---|---|---|
| Temperature | 37° | C. | Fixed |
| pO₂ | 40% | | Cascade (gas mix only) |
| Stirrer | 75 | min⁻¹ | Fixed |
| Air flow | 0.04 | VVM | Fixed |

| | | |
|---|---|---|
| Gas mix flow | variable | Cascade (N_{2;} O_{2;} air) |

After 48 hours of cultivation, a constant addition of glucose was started. A solution of 200 g/L was pumped with 0.42 mL/h until the end of cultivation. After 8 days from the start, the cultivation was finished. In addition to glucose supplementation, an yeast extract (FOR-MS.126) and GlutaMAX (ThermoFisher Lots 2832299—Cat. No: 35050061) solutions were added to final concentrations of 1% and 8 mM, respectively. This supplementation was performed on days 3 and 6 of cultivation.

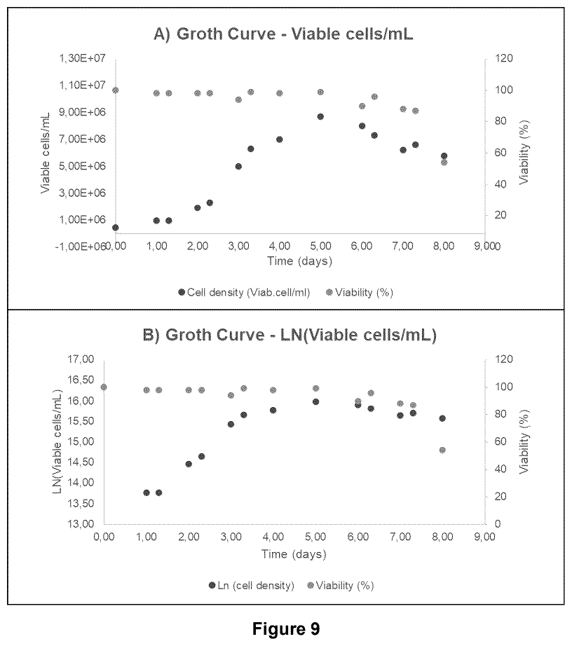

The product was clarified by a first deep filtration using Millistak+HC Pro Pod 0,027 m² ¼ in. Hose Barb. The clarified product was sent to purification. The bioreactor production was carried out for 8 days. Cell growth was monitored throughout the process, and the growth curve can be evaluated below (FIG. 9).

The growth curve showed exponential growth until day 3. Cultivation started a maintenance phase that could be detected on day 5 and, after day 6, it is possible to see a death phase. Cell viability started to drop after day 6 also. Maximum cell density was reached on day 5. One possible explanation for this cultivation behavior is the presence of yeast extract. Other experiments (not shown) have shown that while yeast extract appears to disrupt the growth rate, it enhances protein expression.

1.3.6. Purification from Expression Test

The cultivation was finalized and centrifuged (CEN-001) at 10.000 g at 4° C. by 10 minutes. The supernatant was filtered with Millex PVDF 0.45 μm syringe filter. Samples were collected for Bradford quantitation, SDS-page and ELISA analysis. The supernatant was injected to the Mabselect PrismA Hitrap Column with Akta Pure (FPL-001) at 0.5 ml/min. The column was previously equilibrated with five column volumes of Equilibrium Buffer (Sodium phosphate 20 mM, Sodium Chloride 150 mM, pH 7.2—FOR-MS.044 lot 002/24) at 1 ml/min. The flowthrough was collected at only one fraction. After injection the column was equilibrated with five column volumes of Equilibrium Buffer at 1 ml/min. Elution of nanobody was performed at 1 ml/min by lowing the pH with Elution Buffer (Sodium citrate, pH 3,5—FOR-MS.045 lot 002/24) and the protein was collected and added with Neutralization Buffer (Tris 1 M, ph 8.0—FOR-MS.046 lot 002/24) at a proportion of 1:0.32 ml (eluted volume: neutralization buffer) to adjust the pH close to neutrality. The eluted material was concentrated using a VivaSpin concentrator (3 kDa) previously washed with Sodium Phosphate Buffer (20 mM, pH 7.4—FOR-MS.048 lot 004/24). After all volume was added and concentrated to approximately 2 ml, the diafiltration was performed by adding 8 ml of Sodium Phosphate Buffer and solution was concentrated approximately 2 ml. All cycles of concentration were performed at 6.000 g and at 4° C. (CEN-001). The concentrated material (retentate phase) was then sterile filtered at a biosafety cabinet and stored at 2-8° C.

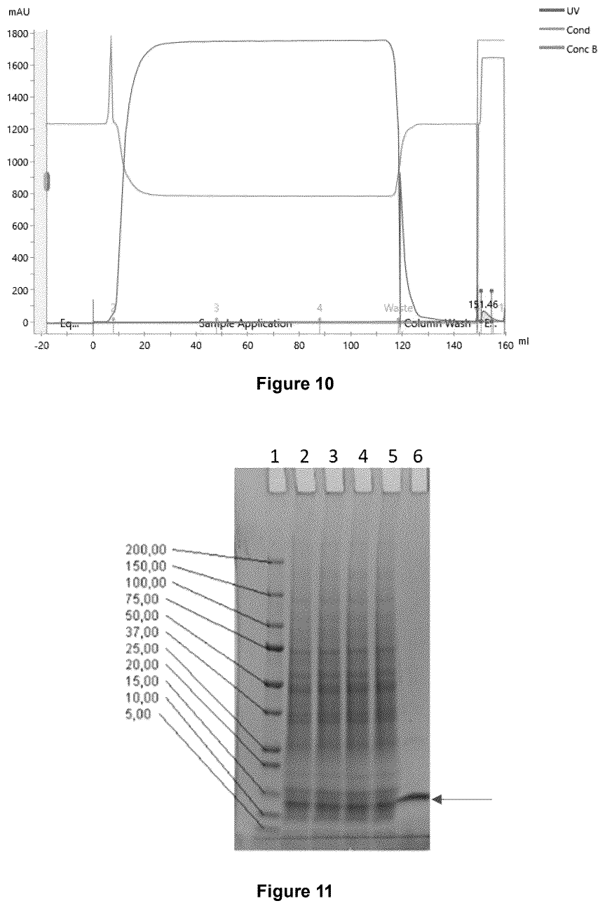

An amount of 113 ml of cultivation supernatant was injected at the PrismA column (FIG. 10). A peak was eluted and collected containing 2.5 ml (containing already Tris 1 M pH 8.0 to neutralize pH).

The SDS-PAGE analysis (FIG. 11) has shown that capture of the nanobody was reached with a high purity, 87%. The band found at the elution fraction (red arrow) has a molecular weight near 15.2 kDa. Quantitation of the eluted material was performed by Bradford method, which results in a concentration of 0.06 mg/ml.

The final concentration and buffer exchange was performed. Diafiltration volume of 5× was used. The final concentration volume was approximately 2.5 ml. The concentrate was sterile filtered (Minisart 0.22 um PES) at a biosafety cabinet, sampled and stored at 2-8° C. The final concentration by Bradford method was 0.06 mg/ml in 1.8 ml solution (lot P005/24).

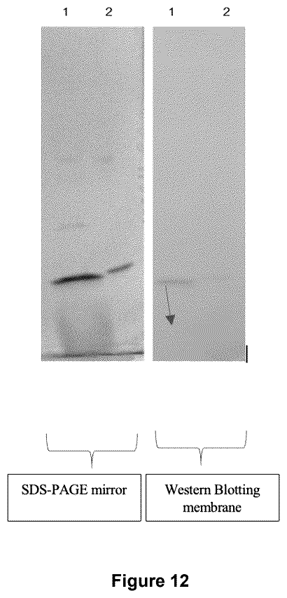

Western Blotting were performed at the elution peak the presence of the nanobody. It was possible to see a positive sign of the nanobody (FIG. 12—red arrow). ELISA analysis was performed at the final product (Sterile concentrated nanobody) in a quantity of 5.76 ng/ml.

1.3.7. Purification from Bioreactor Production

The clarified supernatant was filtered using a Millex syringe filter 0.45 μm. The supernatant was injected into the Sartobind membrane with Akta Pure (FPL-001) at 6 ml/min. The membrane was previously equilibrated with five membrane volumes (MV) of Protein A Equilibrium Buffer (Sodium phosphate 20 mM, Sodium Chloride 150 mM, pH 7.2 at 10 ml/min. The flowthrough was collected at only one fraction. After injection the column was equilibrated with five column volumes of Equilibrium Buffer at 10 ml/min. Elution of nanobody was performed at 10 ml/min by lowing the pH with Protein A Elution Buffer (Sodium citrate, pH 3.5) and the protein was collected and added with Neutralization Buffer (Tris 1 M, pH 8.0) at a proportion of 1:0.32 ml (eluted volume:neutralization buffer) to adjust the pH close to neutrality,

The eluted material was concentrated using VivaSpin concentrator (3 kDa) previously washed with Sodium Phosphate Buffer (20 mM, pH 7.4). After all volume was added and concentrated to approximately 2 ml, the diafiltration was performed by adding seven times the concentrated volume of Sodium Phosphate Buffer and solution was concentrated approximately 1-2 ml. All cycles of concentration were performed in centrifuge at 6.000 g and at 4° C. (CEN-001).

During the night, an issue occurred during the cultivation injection, causing the process to pause after approximately 120 mL had been injected into the Protein A chromatography. This issue was only identified in the next day. As a result, the material remained stationary for approximately 12 hours.

Subsequently, the chromatography process continued with the re-equilibration and elution steps using the already injected volume. To expedite the injection of the remaining volume, the Sartobind Rapid A chromatographic membrane (1.2 mL, Sartorius) was installed and prepared. The only operational difference was the injection flow rate (6 mL/min) and the flow rate of the remaining steps (10 mL/min).

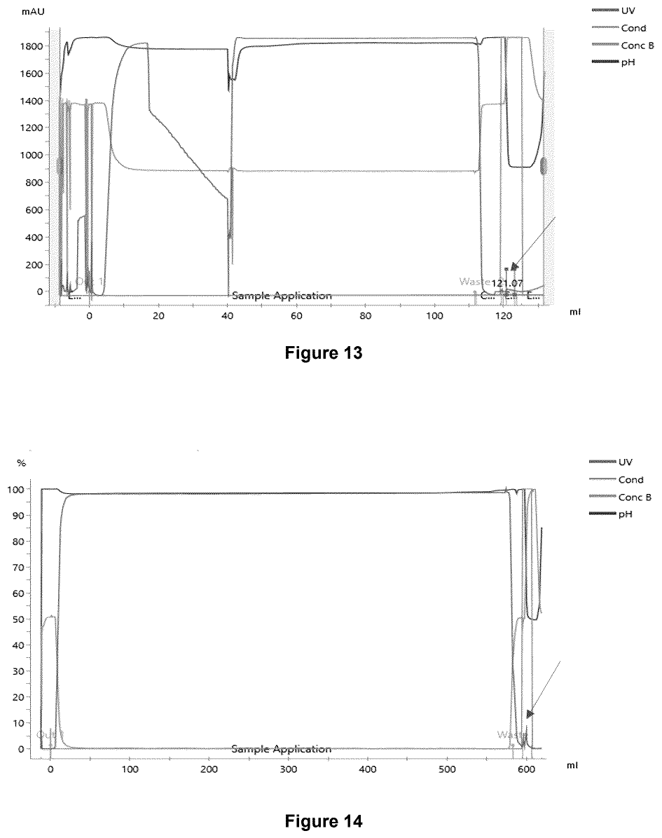

For PrismA Chromatography, an amount of 111 ml of cultivation supernatant was injected at the PrismA column (FIG. 13). A peak was eluted and collected containing 2.9 ml adding Tris 1 M pH 8.0 to neutralize pH to a final volume of 3.8 ml. For Sartobind Rapid A Chromatography, an amount of 582 ml of cultivation supernatant was injected at the Sartobind membrane (FIG. 14). A peak was eluted and collected containing 11 ml adding Tris 1M pH 8.0 to neutralize pH to a final volume of 14.5 ml.

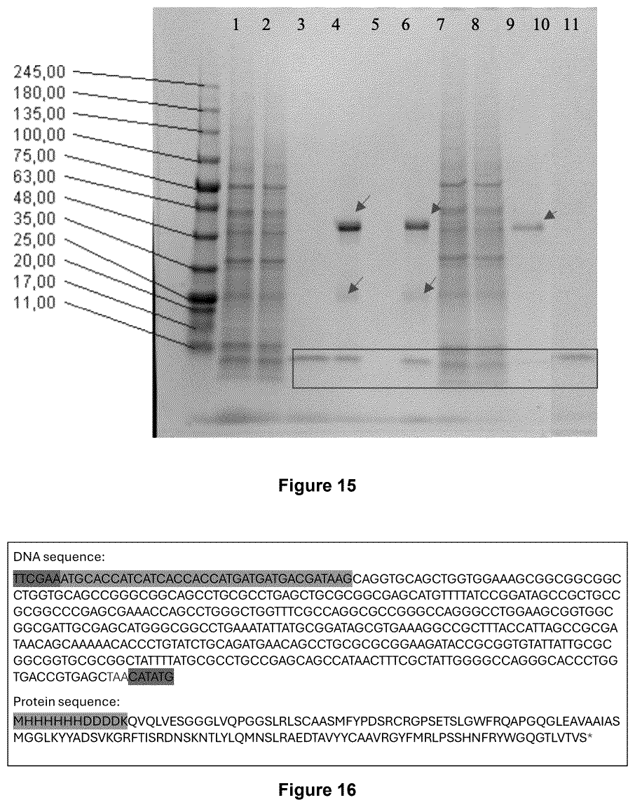

The SDS-PAGE analysis (FIG. 15) has shown that capture of nanobody was reached even with the issues during the night (red lines represents eluted nanobody protein). However, it causes a high quantity of aggregates (blue arrows) that increases in the concentration step, reducing the yield of the process.

Quantitation of the eluted material was performed by Bradford method, which results in a concentration of 0.22 mg/ml for PrismA elution and for Sartobind elution the concentration was above 0.05 mg/ml.

The final concentration and buffer exchange was performed for both PrismA and Sartobind elution. Diafiltration volume of 7× was used at 2 ml concentrated solution (using 20 mM Sodium Phosphate Buffer, pH 7.4). The final concentration volume was approximately 2 ml. The PrismA retentate was sterile filtered with a 0.22 μm syringe filter at a biosafety cabinet, sampled and stored at 2-8° C. (Sartobind did not proceed in the process due to low concentration). The final concentration by Bradford method was 1.36 mg/ml for PrismA in 1.5 ml of solution (Lot LB0052024).

### Example 2—E. coli Cell Line Development

### 2.1.1 Construction of Expression Plasmids (in Silico)

Specifically concerning the construction of the E. coli expression system, pET24a vector map was checked to design the best restriction enzymes. Bst-BI and Nde-I were respectively chosen for upstream and downstream vector cloning. The synthesized sequence underwent codon optimization (Escherichia coli) avoiding the Bst-BI and Not-I restriction sites.

The Genscript package of services provides a proprietary algorithm to codon optimization. The output of the software was the synthesized sequence. FIG. 16 shows the sequence of DNA suggested by Genscript and the in silico translation.

E. coli Cells Cloning

BL21 (DE3) E coli cells (BL21 One shot (DE3)—Thermofisher lot 3438649) were transformed with the vector pET24a_nanobody by heat shock protocol, according to the manufacturer's recommendations. Equipment used in these steps were biosafety cabin (PACHANE—Model PA400); micropipette set and water bath (CapLab—model SSD 5L). The transformed suspension was inoculated onto Petri dishes prepared with LB agar medium (Lot 019/23—FOR-MS.031) supplemented with kanamycin sulfate (Lot 2556471). Incubation was carried out at 37° C. for 18 hours in bacterial Incubator (SPLABOR—SP 101/30.1).

Positive colonies were cultured in LB Broth (FOR-MS.006) and a Research Cell Bank (RCB) was produced by freezing the culture in a 20% glycerol (Dinâmica lot 111836) solution. The RCB was stored at −80° C. (Ultra freezer ULF-001).

### Expression Test

Expression tests were carried out by seeding 150 μL of cryopreserved cells in 30 mL of CD Bacto Supreme (Gibco Lot 2558581) in a 125 mL Erlenmeyer. The cells were cultivated at 37° C., 250 rpm in a Shaker incubator ISR-001 (Solab—model SL-233) for 18 hours. Then, 30 mL of culture were transferred to 500 mL of CD Bacto Supreme (Gibco Lot 2558581) in a 2000 mL Erlenmeyer. Cells were cultivated at 37° C., 250 rpm in a Shaker incubator ISR-001 (Solab—model SL-233) for until OD reach 0.8. The culture was then supplemented with 1 mM IPTG to induce the nanobody production. The cells were cultivated at 37° C., 250 rpm in a Shaker incubator ISR-001 (Solab —model SL-233) for 24 hours. After that all the product was centrifuged by 4000 g in 10 minutes. Pellet cells were twice washed with 200 mL of purified water and then was sent to downstream processing.

### Refolding Purification of Inclusion Bodies

The frozen cells from both cultures were thawed. For each culture, the cells were homogenized with a glass rod, and 120 ml of Lysis Buffer (Tris 20 mM, NaCl 1.5 M, EDTA 0.1 mM, pH 9) were added. Both flasks were well homogenized and transferred to 250 ml beakers. For each beaker, the following sonication cycle was performed in an ice bath, for 8 cycles: 30s ON, 59s OFF, 50% power. A sample of 0.2 ml was performed at cycles 2, 4, and 6. The samples were centrifuged in a mini-centrifuge for 5 minutes. The supernatant was discarded, and the pellet was resuspended in 0.2 ml of purified water, followed by sampling. Centrifugation was performed at 10,000 g for 20 minutes at 5° C. The supernatant was sampled and discarded. The pellet was weighed.

In each flask, 120 ml of Washing Buffer 1 (Tris 20 mM, NaCl 1.5 mM, EDTA 5 mM, Triton X-100 1% v/v, pH 9) was added to the pellet, shaken vigorously, and a sample was collected. The suspension was kept under agitation in an ice bath for 30 minutes. Centrifugation was performed at 10,000 g for 20 minutes at 5° C. The supernatants were sampled and discarded. The pellets were weighed.

In each flask, 120 ml of Washing Buffer 2 (Tris 20 mM, NaCl 1.5 mM, EDTA 5 mM, Urea 2 M, pH 9) was added to the pellet, shaken vigorously, and a 100 μl sample was collected. The suspension was kept under agitation in an ice bath for 30 minutes. Centrifugation was performed at 10,000 g for 20 minutes at 5° C. The supernatants were sampled and discarded. The pellets were weighed.

In each flask, 120 ml of Washing Buffer 3 (Tris 20 mM, EDTA 5 mM, pH 9) was added to the pellet, shaken vigorously, and a sample was collected. The suspension was kept under agitation in an ice bath for 30 minutes. Centrifugation was performed at 10,000 g for 20 minutes at 5° C. The supernatants were sampled and discarded. The pellets were weighed.

An amount of twice the pellet mass of Washing Buffer 3 was added. The suspension was weighed. The OD at 600 nm of the suspension was determined. The inclusion body suspension (IBS) was fractionated into appropriately sized portions and stored at freezing temperature (FT).

An amount of 5 ml of inclusion body suspension was thawed at room temperature and added, under stirring, into a 50 ml beaker containing 15 ml of Solubilization Buffer (Sodium Phosphate 20 mM, Dithiothreitol 300 mM, Urea 6.6 M, pH 12). The pH was checked and adjusted to 12.0±0.2, and stirring was maintained for 15 minutes. A 0.5 ml aliquot of the solubilized solution was sampled and centrifuged at 10,000 g in 10 minutes.

The supernatant was separated as a sample, and 0.5 ml of water was added to the pellet. The sample was resuspended and stored.

The solubilized solution was filtered through a 0.45 μm PES syringe filter (with a glass fiber pre-filter), and 12 ml of the filtered solution was added to 108 ml of Refolding Buffer (Tris 20 mM, NaCl 50 mM, sucrose 100 mM, glycerol 100 mM, Tween20 0.1% v/v, Triton X-100 0.1% v/v, pH 8), mixing gently in an ice bath.

The refolding solution was filtered through a 0.45 μm PES syringe filter (with a glass fiber pre-filter), and an aliquot was collected. The solution was kept refrigerated overnight. Anion Exchange Chromatography was performed using Praesto Jetted Q65 (5 ml). The injection flow rate was set to 2.0 ml/min, while the buffer flow rate was 3 ml/min. The column was equilibrated with 5 CV of Sodium Phosphate Buffer (20 mM, pH 7.4). The entire filtered refolding volume was injected while being kept in an ice bath. The flowthrough fraction was collected in samples of at least 50 ml. The column was then re-equilibrated with 5 CV of Sodium Phosphate Buffer (20 mM, pH 7.4). Elution was performed using Sodium Phosphate Buffer (20 mM, pH 7.4) with a gradient ranging from 0 to 100% of Washing Buffer (20 mM Sodium Phosphate, 1 M Sodium Chloride, pH 7.4) over 30 column volumes. Collection started after the UV reading exceeded 10 mAU, and fractions were collected in volumes of 1 to 5 ml.

### Protein Production

The production in the E. coli expression system was carried out in the following stages: pre-inoculum, inoculum, cell growth phase, and expression phase (IPTG induction). After that, the material was centrifuged, and the pellet was used for purification.

### Refolding Purification of Inclusion Bodies

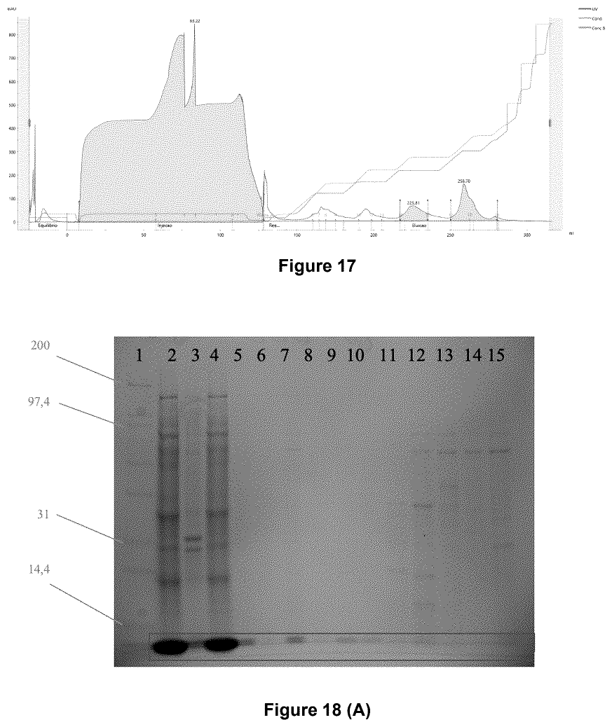

After recovery, solubilization of inclusion bodies occurred at a pH range between 11.50 and 12.22. After addition to the refolding buffer, the initial pH was 9.28 and was adjusted to 8.05. The refolding solution was then filtered and left to rest for 16 hours at a temperature of 2-8° C. A total volume of 120 mL was injected into the Praesto Q65 5 mL column (Purolite), which had been pre-equilibrated with equilibrium buffer (FIG. 17). After re-equilibration, during the gradient from 0 to 2 M NaCl, the formation of peaks was monitored. The gradient was stopped as soon as the peaks started to form and was resumed once the peaks had fully eluted. The peaks were collected in fractions as they were being eluted.

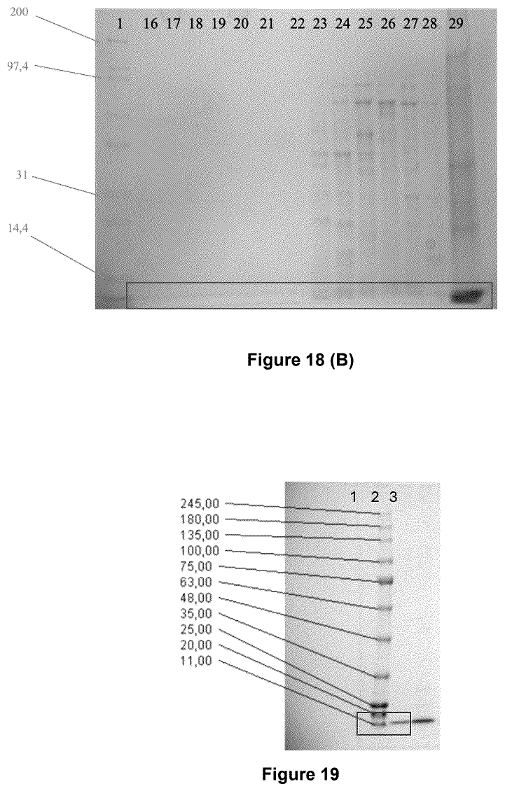

The SDS-PAGE analysis (FIGS. 18 and 19) showed that the fractions with the purest protein of interest came from the flowthrough. The column retained most of the impurities, and a large part of the material did not bind. Most of the elution fractions came out with many impurities or were overly diluted. Thus, the concentration of one of the flowthrough fractions (F4) was performed.

The concentration and buffer of exchange of F4 flowthrough fraction was performed with VivaSpin 10 kDa. Diafiltration volume of 6× was used with Tris 20 mM pH 8.5. The final concentration volume was approximately 0.8 ml. The concentrate was sterile filtered (Minisart 0.22 μm PES) at a biosafety cabinet, sampled and stored at 2-8° C. The final concentration by Bradford method was 0.36 mg/ml in 0.6 ml solution (lot P005/24). FIG. 19 shows the SDS-PAGE of the final material that indicated a purity of 80.5%. Thus, the final concentration considering this purity was 0.26 mg/ml.

Pichia pastoris Cell Line Development

### Construction of Expression Plasmid

The team devised cloning strategies to direct expression in three different expression systems: E. coli, P pastoris, and CHO cells. Specifically concerning the construction of P pastoris expression system, pPICZ-A vector map was checked to design best cloning strategy. A signal peptide known as “Gallus factor” was chosen (MLGKNDPMCLVLVLLGLTALLGICQG). Bst-BI and Not-I were respectively chosen for upstream and downstream vector cloning. The synthesized sequence underwent codon optimization (Pichia pastoris) avoiding the Bst-BI, Not-I and Sac-I (applied on linearization step) restriction sites.

### Construction of Expression Plasmids (in Silico)

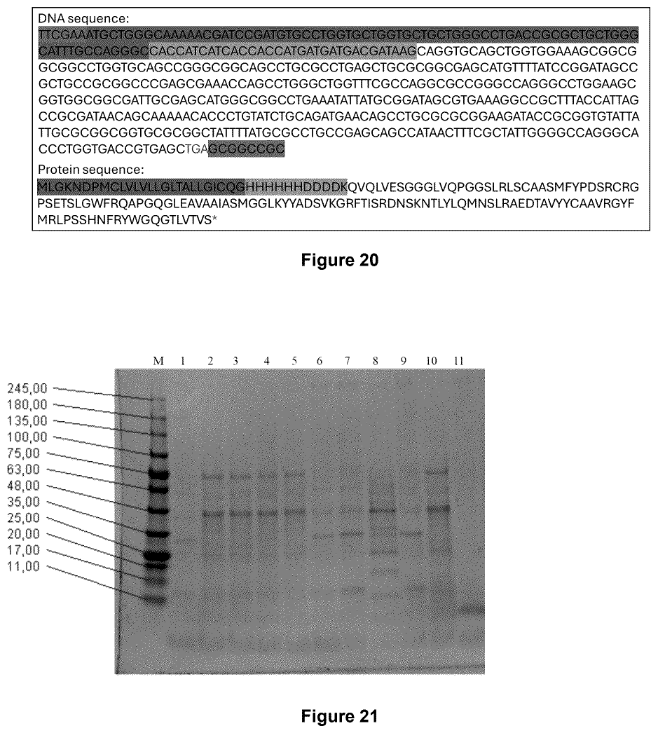

The Genscript package of services provides a proprietary algorithm to codon optimization. The output of the software was the synthesized sequence. FIG. 20 shows the sequence of DNA suggested by Genscript and the in silico translation.

### DNA Preparation for Electroporation

With the plasmid in hand, the team conducted procedures for replicating the material to achieve the necessary quantity for electroporation; linearization to increase the recombination and ensure efficiency of the integration of the insert to yeast genome; and purification to enhance the chances of success in the event.

Transformation in E. coli

The initial step in preparing the DNA used in the project involved transforming E. coli bacteria, capable of replicating the plasmid. “One Shot™ TOP10 Chemically Competent E. coli” cells (26077075B) were used. Transformations were performed using a heat shock method according to the manufacturer's recommendations (Invitrogen—MAN0000633). Equipment used in these steps were biosafety cabin (PACHANE—Model PA400); micropipette set and water bath (CapLab—model SSD 5L). The transformed suspension was inoculated onto Petri dishes prepared with LB agar medium (Lot 019/23—FOR-MS.031) supplemented with Zeocin (Gibco Lot 2614510). Incubation was carried out at 37° C. for 18 hours in bacterial Incubator (SPLABOR—SP 101/30.1).

### DNA Extraction

Following successful bacterial transformations, the next step was to extract plasmid DNA in sufficient quantity for subsequent stages. For this purpose, the “PureYield Plasmid Midiprep System” kit from Promega (Lot 587624) was used as per the manufacturer's recommendations (Promega—TM253). The extracted material was quantified using spectrophotometry (A=260 nm) with a Tecan instrument, using the Nanoquant tool (Infinite Pro 200). The equipment reads at A=260 nm, and the “260/280” ratio was used as a quality parameter for the solution. Extractions were deemed acceptable for “260/280” ratios greater than or equal to 1.8.

### Plasmid Linearization

With the material in adequate quantity and quality, the next step was plasmid linearization. The enzyme Sac-I (New England Biolabs—Lot 507132) was used according to the manufacturer's recommendations. Quality analysis of the linearized material was performed by agarose gel electrophoresis at 0.8% (Fastbio Tablets—Lot D0057). 1 kb DNA LadderGene Ruler (LOT SLCK0031) was used as the molecular weight standard. Electrophoresis was carried out with the equipment from KASVI (model K3316H).

### DNA Purification

Upon successful confirmation of the linearization reaction, the material underwent a DNA precipitation-based purification process. For this, 1/10 volume of 3 M sodium acetate solution (Dinâmica—Lot 111148) was added to each tube containing the linearization product, followed by gentle homogenization. Subsequently, 2.5 times the volume of 96% ethanol (Sigma—Lot 1274427308) was added, followed by homogenization and incubation at −20° C. in freezer (Consul—model CRD37E) for approximately 18 hours. After this period, the material was centrifuged at 12,000 g, 4° C. for 45 minutes. The pellet was then washed twice with 70% ethanol. After the second wash, the pellet was incubated at 37° C. to completely evaporate the ethanol. Finally, the pellet was resuspended in ultrapure water. The material was quantified again, as described in section 4.2.2.2, following the same quality criteria.

P pastoris Cells Cloning

P pastoris cell cloning starts with an electroporation followed by plate spread to isolate colonies (clones). After that, a screening based on productivity is carried out to find best clones for Nanobody production.

### Electroporation

The electroporation protocol begins with preparing a liquid yeast culture until it reaches an optical density of 1.3. The cells were washed twice with ice-cold water and then resuspended in a 1 M sorbitol solution (Sigma Lot: BCCJ0161). Approximately 1 μg of linearized DNA was added to 40 μL of electrocompetent cells. The electroporation conditions were 1.5 kV, 25 μF, and 200Ω (Electroporator ETP-001 Bio-Rad Micropulser). Liquid YPD (FOR-MS.022) medium was added to allow cell recovery. This solution was plated onto a solid YPDS (YPD+1 M sorbitol) medium supplemented with Zeocin (Gibco Lot 2614510). After 48 hours, isolated colonies were selected for productive screening.

### Clone Selection Screening

Approximately 20 colonies were selected for productive screening. For this purpose, cultures were carried out in two stages: a growth stage with 10 mL of BMGY (FOR-MS.054+10% Glycerol (Dinâmica Lot 111836)) and an expression stage with 7 mL of BMMY (FOR-MS.054+10% Methanol (Dinâmica Lot 123126)). The cultures were performed in 50 mL conical tubes in a shaker at 180 rpm and 30° C. The cultures were maintained for up to 96 hours of expression.

### Expression Test

After selecting a productive colony, a larger-scale culture was conducted to produce material for shipment to the client. For this, 2 L Erlenmeyer flasks were used for the growth stage with 500 mL of BMGY (FOR-MS.054+10% Glycerol (Dinâmica Lot 111836)) and baffled 1 L Erlenmeyer flasks were used for the expression stage with 100 mL of BMMY (FOR-MS.054+10% Methanol (Dinâmica Lot 123126)). The cultures were maintained in a shaker at 180 rpm and 30° C. for up to 96 hours of expression.

After electroporation and clone selection, productive screening was performed to identify the most productive clone. Following the screening cultures, an SDS-PAGE gel was run to evaluate the expression profile of each clone (FIG. 21).

The SDS-PAGE analysis showed that different clones exhibited distinct expression profiles. Protein bands of the expected size (˜15 kDa) were observed for some clones (lanes 7, 8, 9, 10). However, bands representing significantly higher molecular weights were also detected (bands not found in the negative expression control), which may indicate nanobody oligomerization. Given the diversity of expression patterns, four clones were selected for a larger-scale expression test.

The larger-scale expression test was performed as described in section 4.2.4. After the production period, the cultures were centrifuged, and the supernatant was purified as described below.

Purification from Expression Test

The entire volume of the supernatant was filtered using a 0.45 μm Millex syringe filter. Chromatography purification was performed using two columns in series.The first column used was MabSelect PrismA Cytiva (1 ml), and the second column was IMAC-Ni Cytiva (5 ml). The injection flow rate was set to 1.0 ml/min, and the buffer flow rate was also 1.0 ml/min. Equilibration was performed with 5 CV of Equilibrium Buffer (20 mM sodium phosphate, 150 mM sodium chloride, pH 7.2). The entire volume of the supernatant was injected, and the flowthrough fraction was collected in a single fraction. The columns were re-equilibrated with 5 CV of Equilibrium Buffer. The IMAC-Ni column was then disconnected, and the material was eluted through the PrismA column using 10 CV of Elution Buffer (100 mM sodium citrate, pH 3.5). Collection started after an increase in the UV signal. The eluate was collected in 15 ml tubes, transferred into 15 ml centrifuge tubes, and Neutralization Buffer (1 M Tris, pH 8.0) was added at a ratio of 0.320 ml per 1 ml collected.

After collection, an additional 5 CV of Elution Buffer was passed through. The PrismA column was then disconnected, and the IMAC-Ni column was connected. The material was eluted using 10 CV of IMAC Wash Buffer (20 mM Tris, 500 mM sodium chloride, 500 mM imidazole, pH 8.0). Collection started after an increase in the UV signal, and the eluate was collected in 15 ml tubes before being transferred into 15 ml centrifuge tubes.

The column was then washed with 10 CV of IMAC Wash Buffer. After collection, both columns (PrismA followed by IMAC) were reconnected, and an additional 5 CV of FOR-MS.044 buffer was passed through the two columns.

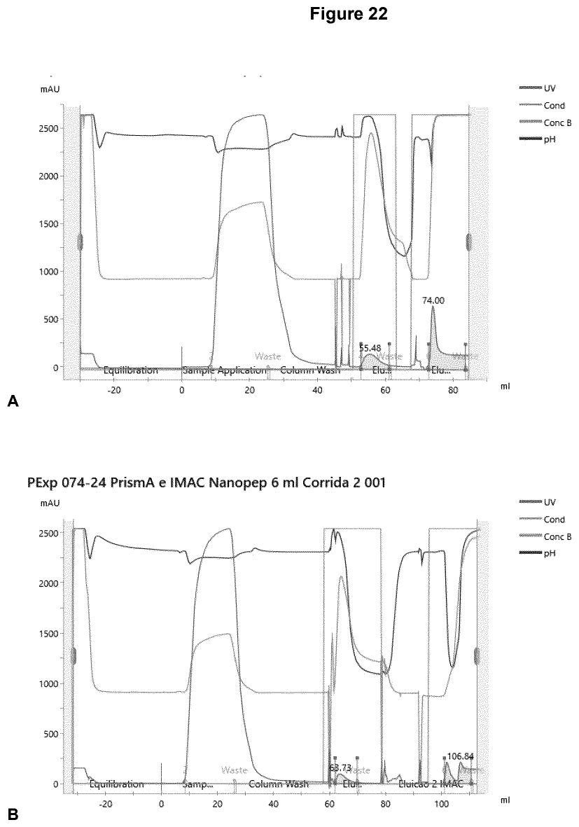

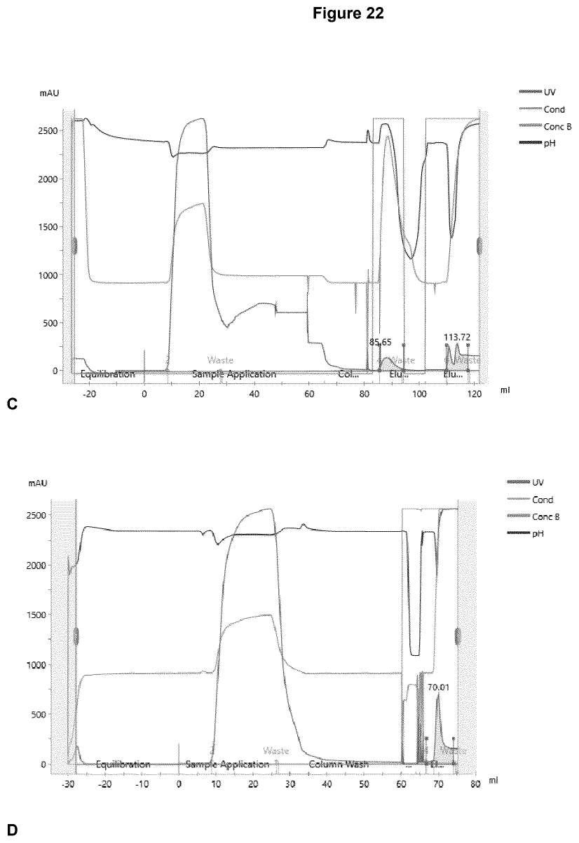

An amount of 25-30 ml of cultivation supernatant of each clone was injected at the PrismA-IMAC columns (FIG. 22). A peak was eluted for each column (except for PrismA clone 30 that did not have a peak) and collected containing 2.5 ml (containing already Tris 1 M pH 8.0 to neutralize pH).

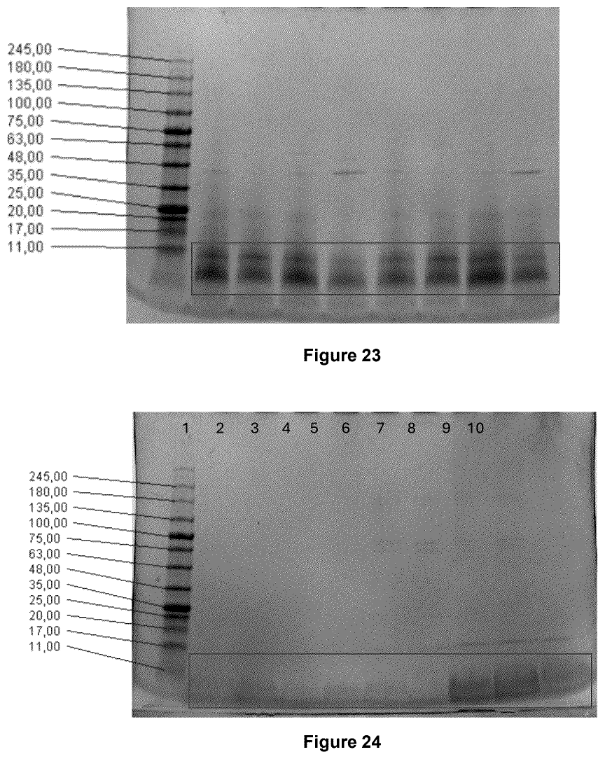

The SDS-PAGE analysis (FIG. 23) has shown that capture of nanobody was reached with a high purity, 87%. The band found at the elution fraction (red arrow) has a molecular weight near 15.2 kDa.

Quantitation of the eluted material was performed by Bradford method, which results in a concentration of 0.06 mg/ml.

The final concentration and buffer exchange was performed. Diafiltration volume of 7× was used. The final concentration volume was approximately 2.5 ml. The concentrate was sterile filtered (Minisart 0.22 μm PES) at a biosafety cabinet, sampled and stored at 2-8° C. The final concentration by Bradford method was 0.06 mg/ml in 1.8 ml solution (lot P005/24).

Analysis of the chromatograms confirmed that most of the material eluted from the IMAC column for all clones. However, all eluates exhibited concentrations below 0.05 mg/mL, as determined by the Bradford method.

To improve quantification by Bradford and allow for SDS-PAGE analysis, the eluates were concentrated using Vivaspin concentrators with a 3 kDa membrane (Sartorius). FIGS. 23 and 24 show the SDS-PAGE analysis for Columns IMAC and ProteinA, respectively and Table 1 the quantification by Bradford method.

SDS-PAGE analysis confirmed a higher quantity of the target protein in the IMAC column eluates. In contrast, the PrismA column yielded at least three times less protein, as an additional concentration step was required (Lanes 8 to 10) to obtain bands of similar intensity to those observed in the IMAC column.

Additionally, Bradford assay results indicated that the protein concentration remained low for both columns.

TABLE 1
Total protein concentration by Bradford Method of final products

| | | Bradford |
|---|---|---|
| | | Concentration |
| Label | Description | (mg/ml) |
| Concentrated Nanobody (called herein | Clone 30 concentrated elution IMAC | 0.07 |
| Nanopep) - Purification 1 | | |
| Concentrated Nanobody (called herein | Clone 21 concentrated elution IMAC | <0.05 |
| Nanopep) - Purification 2 | | |
| Concentrated Nanobody (called herein | Clone 31 concentrated elution IMAC | 0.07 |
| Nanopep) - Purification 3 | | |
| Concentrated Nanobody (called herein | Clone 35 concentrated elution IMAC | <0.05 |
| Nanopep) - Purification 4 | | |
| Concentrated Nanobody (called herein | Clone 21 concentrated elution PrismA | <0.05 |
| Nanopep) - Purification PtnA 2 | | |
| Concentrated Nanobody (called herein | Clone 31 concentrated elution PrismA | <0.05 |
| Nanopep) - Purification PtnA 3 | | |
| Concentrated Nanobody (called herein | Clone 35 concentrated elution PrismA | <0.05 |
| Nanopep) - Purification PtnA 2 | | |

Nanobodies Interaction with a Known Antibody-Protein a Complex

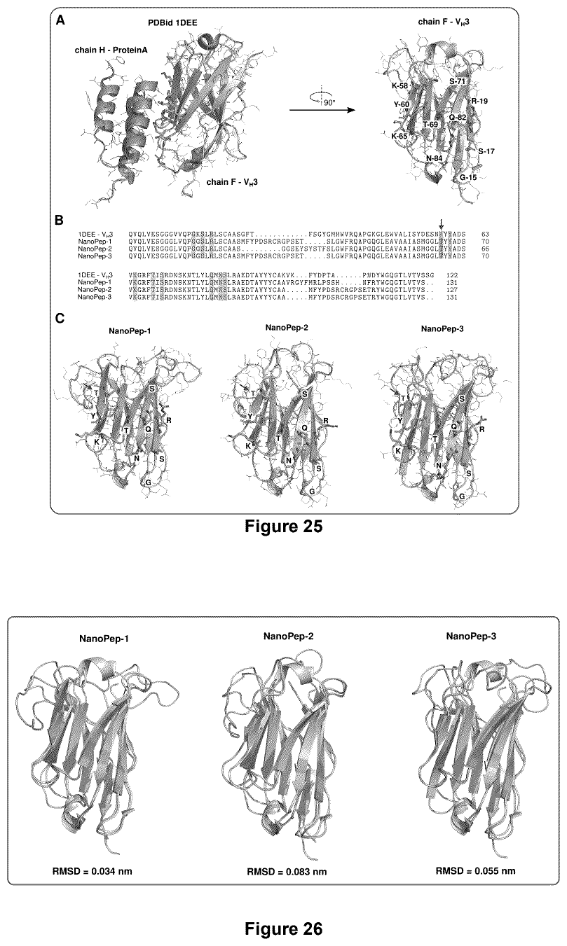

In the first stage of evaluating the potential for nanobodies binding to ProteinA, which comprises the MabSelect PrismA® separation column, we carried out a comparative analysis of the sequences and structures of nanobodies named NanoPeps-1 to -3 with the heavy chain of the conventional antibody (VH3) model whose complex with protein A is very well characterized in the literature (DOI: 10.1073/pnas.97.10.5399) (FIG. 25A—left). The VH3 hotspot residues important for the interaction and formation of a stable complex with ProteinA described in the separation column datasheet (MabSelect PrismA®) were mapped onto the VH3 structure (FIG. 25A—right) and compared in the alignment matrix constructed from the sequences of VH3 and NanoPeps (FIG. 25B). We can only observe a change along the NanoPeps sequence relative to the K-58 position of the VH3 by replacing lysine with a threonine (T). Structurally, we also observed equivalence in the spatial arrangements of hotspots in NanoPeps when compared to VH3 (FIG. 25C).

Additionally, despite the observation of equivalence in the spatial arrangements of hotspots in NanoPeps compared to VH3, we observed small differences in local three-dimensional disposition of #-sheets of framework region which contained these hotspots residues (FIG. 26). This led us to move to a more refined stage for evaluation of the formation of NanoPeps-ProteinA complexes by studying the dynamic behavior using molecular dynamics simulations.

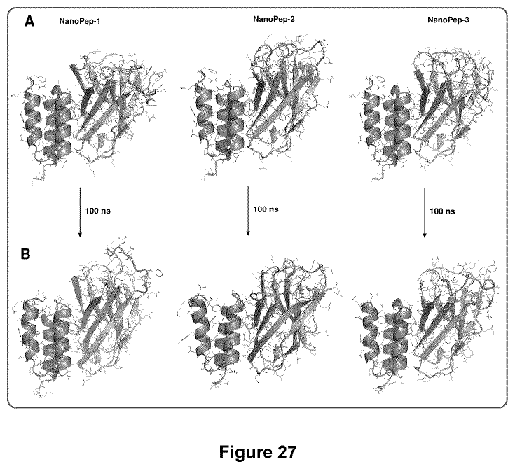

We constructed the NanoPeps-ProteinA complexes by superposition over crystallographic structure of VH3-ProteinA complex (PDBid 1DEE) (FIG. 27A). Despite the presence of threonine (T) in NanoPeps in the position equivalent to lysine (K) in VH3 being considered tolerable for ProteinA binding (MabSelect PrismA®), it is interesting to include the T to K mutation in NanoPeps for evaluation of its benefits in complex formation. For this purpose, we also constructed T to K mutants systems for dynamical evaluation by molecular dynamics. Using these NanoPeps-ProteinA complexes models and VH3-ProteinA as initial models we constructed systems for molecular dynamics simulations in explicit solvent and 150 mM ionic strength, totalizing a total of seven molecular systems for massive simulation. The simulations were carried out with the aid of the GROMACS-2022 software and Amber ff99SB force field, using the simulation protocols detailed in a recently published study (DOI: 10.1080/07391102.2020. 1728385) at 25° C. The simulations were performed for 100 ns at the Lince2 computer cluster allocated at the Information Technology Superintendence of University of Sso Paulo (https://hpc.usp.br/), equipped with GPU computing distributed in 8 cores of an Intel Xeon® E5-server 2680 @2.70 GHz and a Tesla K20m GPU graphics.

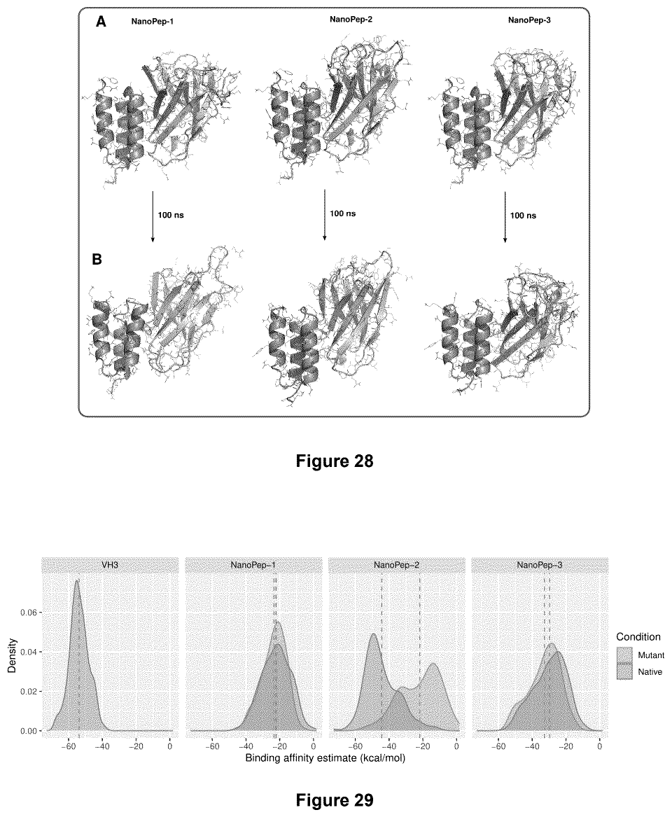

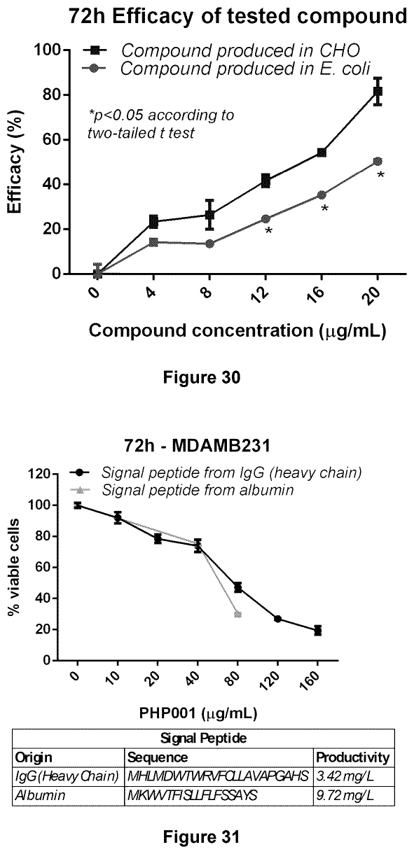

First, the models constructed revealed the absence of steric effects to the formation of NanoPeps-ProteinA complexes (FIG. 27A). The analysis of molecular dynamics generated trajectories showed that after 100 ns the native NanoPeps complexes did not showed major structural changes in relation to the initial models (FIG. 27B). Similar behaviors were observed for the NanoPep-1 and -3 in mutant systems (FIG. 28). However, for the NanoPep-2 mutant we observed a large reorganization relative to initial structure and also for the other systems (FIG. 28), suggesting a strong influence of the K mutation in the formation of this complex. We also evaluated the binding affinity by molecular mechanics generalized Born surface area (MM/GBSA) calculation (DOI: 10.1021/acs.jctc.1c00645, DOI: 10.1021/ct300418h) from trajectories (FIG. 29). In the comparison with VH3 we observed a more attractive binding affinity than for the NanoPeps. However, the maintenance of complex structures and attractive (negative) binding affinity values for NanoPeps indicated potential for maintenance of complexes with ProteinA. Energetically, NanoPep-2 in its native form has a higher binding affinity for ProteinA (˜−44 kcal·mol⁻¹) while the other two NanoPeps have less intense values (between ˜−22 to −30 kcal·mol⁻¹) (FIG. 29 and Table 2). In the case of the mutants, we observed a behavior of slight improvement in the binding affinity for NanoPeps-1 and -3, but, compatible with already observed distortion of its structure, the NanoPep-2 mutant presented lower affinity than its native form (FIG. 29 and Table 2). These results suggested that the mutant form for NanoPeps-1 and -3 are acceptable, but for NanoPep-2 the native form would be better choice for binding to ProteinA.

TABLE 2
Mean binding affinity estimates for complex formation
for VH3 and NanoPeps with Protein A by MM/GBSA Method

Mean binding affinity (kcal · mol⁻¹)

| Complex with protein A | Native | Mutant |
|---|---|---|
| V_{H}3 | −53.7 ± 5.6 | — |
| NanoPep-1 | −22.2 ± 8.4 | −23.4 ± 7.3 |
| NanoPep-2 | −44.3 ± 10.1 | −21.8 ± 12.1 |
| NanoPep-3 | −29.7 ± 9.7 | −32.7 ± 9.2 |

### SEQ ID LIST

SEQ ID 1
QVQLVESGGGLVQPGGSLRLSCAASSLGWFRQAPGQGLEAVAAIASMGG
LKYYADSVKGRFTISRDNSKNTLYLQMNSLRAEDTAVYYCAARYWGQGT
LVTVS
SEQ ID 2
MFYPDSRCRGPSET
SEQ ID 3
CMFYPDSRCRGPSETC
(linear)
SEQ ID 4
MFYPDSR
SEQ ID 5
CRGPSET
SEQ ID 6
CMFYPDSRC
(linear)
SEQ ID 7
CRGPSETC
(linear)
SEQ ID 8
MFYPDSRSRGPSET
SEQ ID 9
MDWTWRVFCLLAVAPGAHS

What is claimed is:

1. A nanobody platform comprising a single-domain antigen-binding fragment of humanized camelid heavy-chain antibody having a protein A binding site.

2. The nanobody platform according to claim 1, wherein the nanobody has at least 70% similarity with the SEQ ID 1.

3. The nanobody platform according to claim 1 having at least one of the following features in its amino acid sequence selected from the group consisting of: i) an interval between the S25 and S26 amino acids for peptide insertion; ii) an interval between the A91 and R92 amino acids for peptide insertion; and iii) the presence of the K amino acid in the 51 position.

4. The nanobody platform according to claim 2, wherein the nanobody or peptide-modified nanobody incorporates a peptide in the CDR1 region located between the S25 and S26 amino acids of the nanobody having at least 70% of similarity with the SEQ ID 1.

5. The nanobody platform according to claim 2, wherein the nanobody or peptide-modified nanobody incorporates a peptide in the CDR3 region located between the A91 and R92 amino acids of the nanobody or peptide-modified nanobody has at least 70% of similarity with the SEQ ID 1.

6. The nanobody platform according to any one of claim 1, wherein the nanobody or peptide-modified nanobody incorporates a peptide in the CDR1 and/or CDR3 region of the nanobody.

7. The nanobody platform according to claim 1, wherein the nanobody comprises a single-domain antigen-binding fragment of humanized camelid heavy-chain antibody having a purity greater than 80%.

8. A peptide-modified nanobody comprising a single-domain antigen-binding fragment of humanized camelid heavy-chain antibody having a protein A binding site and incorporating a bioactive peptide.

9. The peptide-modified nanobody according to claim 8, wherein the bioactive peptide is a non-toxic bioactive snake venom peptide.

10. The peptide-modified nanobody according to claim 8, wherein the nanobody has at least 70% of similarity with the SEQ ID 1.

11. The peptide-modified nanobody according to claim 8, wherein the nanobody has at least 70% of similarity with the SEQ ID 1 and the peptide is selected from the group consisting of at least 70% of similarity with the SEQ ID 2, SEQ ID 3, SEQ ID 4, SEQ ID 5, SEQ ID 6, SEQ ID 7 and SEQ ID 8.

12. The peptide-modified nanobody according to claim 8, wherein the peptide-modified nanobody is therapeutically effective for the treatment of solid and non-solid tumors.

13. The peptide-modified nanobody according to claim 8, wherein the peptide-modified nanobody is therapeutically effective against breast, lung, prostate, colon, skin, brain, pancreas or kidney cancer.

14. The peptide-modified nanobody according to claim 8, wherein the peptide-modified nanobody is therapeutically effective for the treatment of triple negative breast cancer.

15. The peptide-modified nanobody according to claim 8, wherein the nanobody comprises a single-domain antigen-binding fragment of humanized camelid heavy-chain antibody having a purity greater than 80%.

16. A production process for a nanobody or a peptide-modified nanobody, comprising the following steps:
a) Construction of systems for expression control and amplification;
b) Purification and eukaryotic cells transformation; and
c) Clone selection, nanobody expression and purification.

17. The production process according to claim 16, wherein the expression control comprises plasmids comprises CHO cells, E. coli and/or P pastoris.

18. The production process according to claim 16, wherein it comprises a large-scale production capacity of nanobody or peptide-modified nanobody.

19. The production process according to claim 16, wherein the peptide-modified nanobody comprises a non-toxic bioactive snake venom peptide.

20. A method for the treatment, prevention or diagnosis of patients, the method comprising using a nanobody platform according to claim 1.

21. A method for the treatment, prevention or diagnosis of patients, the method comprising using a peptide-modified nanobody according to claim 8.
